(19) United States
(12) Patent Application Publication
(10) Pub. No.: US 2025/0009716 A1
(43) Pub. Date: Jan. 9, 2025

(54) NOVEL INHIBITORS OF CLAUDIN-1 FOR THERAPEUTIC INTERVENTIONS

(71) Applicant: BOARD OF REGENTS OF THE UNIVERSITY OF NEBRASKA, Lincoln, NE (US)

(72) Inventors: Punita Dhawan, Omaha, NE (US); Corey Hopkins, Omaha, NE (US); Amar Singh, Omaha, NE (US); Iram Fatima, Omaha, NE (US)

(21) Appl. No.: 18/711,139

(22) PCT Filed: Nov. 18, 2022
(86) PCT No.: PCT/US22/50429

§ 371 Date: May 17, 2024

Related U.S. Application Data
(60) Provisional application No. 63/280,704, filed on Nov. 18, 2021.

(51) Int. Cl. A61K 31/423; A61K 31/4184; A61P 35/00; C07D 235/14; C07D 263/57

(52) U.S. Cl. CPC A61K 31/423; A61K 31/4184; A61P 35/00; C07D 235/14; C07D 263/57

(57) ABSTRACT

Provided herein are compounds and methods for modulating claudin-1 (CLDN1). More particularly, provided are inhibitors claudin-1 and the uses of such inhibitors in regulating diseases and disorders, e.g., to treat cancer.

### CROSS-REFERENCE TO RELATED APPLICATION

The benefit of priority to U.S. Provisional Patent Application No. 63/280,704 filed Nov. 18, 2021, is hereby claimed and the disclosure is incorporated herein by reference in its entirety.

### STATEMENT OF U.S. GOVERNMENT SUPPORT

This invention was made with government support under grant 101 BX002086 awarded by the National Institutes of Health. The government has certain rights in the invention.

### BACKGROUND

Despite the advances made in diagnosis and targeted therapy, the 5-year survival rate for stage-IV colon cancer patients remains a meager 13% (12). Unfortunately, nearly ⅔ of the newly diagnosed colorectal cancer (CRC) patients demonstrate metastasis to distant organs at the time of cancer diagnosis (13). There are no effective means to prevent or delay CRC metastasis and few effective treatment options exist once metastasis has occurred. Therefore, for effective clinical management and to overcome the limitations of standard chemotherapy, development of novel drug targets is urgent. There is accordingly a need for new therapies for CRC progression and metastasis.

Since association of claudin-1 with proto-oncogenes Src is modifiable, it is a promising strategy for prophylactic preventative therapy for at-risk individuals, including patients with strong family histories of aggressive colon cancer or those harboring high claudin-1 expression in their tumors. Preclinical results suggest efficacy in targeted anti-claudin-1 therapy aimed at eradicating the potential of chemoresistance and/or adjuvant therapy with conventional anti-CRC therapies. Thus, claudin-1 inhibition may have a substantial impact on prevention/inhibition of CRC progression and metastasis.

Dysregulated claudin-1 expression in collaborating with Src influences CRC progression and metastasis. However, the mechanism(s) by which claudin-1 expression promotes resistance to anoikis and CRC metastasis are uncertain. It is therefore desirable to have a clear mechanistic understanding of the how claudin-1 inhibitors regulate colon cancer malignancy, and thus new scientific information on the drivers of CRC metastasis and chemoresistance.

Despite the clear knowledge of the role of claudin-1 in promoting CRC metastasis, there is no available inhibitor of claudin-1. It is therefore desirable to (i) develop novel claudin-1 inhibitors for CRC progression and metastasis, (ii) develop biomarkers capable of risk stratification and (iii) develop novel therapeutics. Further, many traditional chemotherapeutic agents suffer from a narrow therapeutic index that presents as a major drawback in treating cancer, in addition to a lack of specificity, severe side effects, and development of drug resistance. Therefore, compounds that inhibit antiangiogenesis and/or target mitochondrial oxidative metabolism are appealing targets for treating tumors and assisting in overcoming the side effects of the chemotherapeutic agents.

### SUMMARY

Provided herein are compounds and methods for inhibiting claudin-1 (CLDN1), and which can treat or prevent a disease or disorder associated with claudin-1 in a subject, such as cancer.

In one aspect, the disclosure provides compounds having the structure of Formula I:

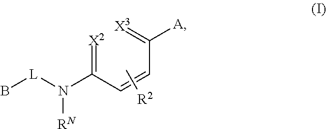

wherein B is C₆-C₁₀ aryl or 5-10 membered heteroaryl comprising 1-3 ring heteroatoms independently selected from O, S, and N, wherein the aryl or heteroaryl is substituted with one R¹; X¹ is CH or N; X² is CH or N; X³ is CH or N; L is

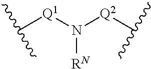

or 5-membered heteroaryl comprising 1 to 3 heteroatoms independently selected from O, S, and N; Q¹ is a bond, C═O, or C═S; Q² is C═O, C═S, or SO₂; A is C₃-C₁₀ cycloalkyl, 3-10 membered heterocycloalkyl comprising 1-3 ring heteroatoms independently selected from O, S, and N, C₆-C₁₀ aryl, or 5-10 membered heteroaryl comprising 1-3 ring heteroatoms independently selected from O, S, and N, wherein the cycloalkyl, heterocycloalkyl, aryl, and heteroaryl are optionally substituted with 1-3 R³; R¹, R², and R³ are each independently H, halo, OH, CN, C₁₋₆ alkyl, C₁₋₆ haloalkyl, C₁₋₆ hydroxyalkyl, C₁₋₆ alkoxy, or C₁₋₆ haloalkoxy; and each R^{N} is independently H or C₁₋₆ alkyl, or two R^{N} together with the atoms to which they are attached form a 5-6 membered heterocycloalkyl ring further comprising 0 or 1 additional ring heteroatoms selected from O, S, and N. In some aspects, the compound has the structure of Formula Ia or Ib:

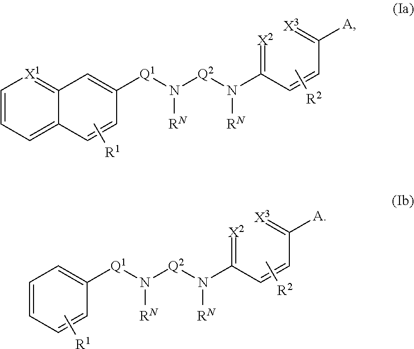

Also provided are compounds as disclosed in Table 1. Further provided are methods of using the compounds of Formula I, Ia, Ib, and Table 1 for inhibiting claudin-1, and/or for treating or preventing diseases and disorders as disclosed herein. Other aspects of the disclosure include a compound as disclosed herein for use in treating or preventing a disease or disorder associated with claudin-1, and the use of a compound as disclosed herein for use in the preparation of a medicament for treating or preventing a disease or disorder associated with claudin-1, such as cancer.

### BRIEF DESCRIPTION OF THE DRAWINGS

FIG. 1 shows that claudin-1 binds with Src in a complex. (A) Co—IP using anti-claudin-1 antibody followed by immunoblotting using anti-Src, claudn-1 antibody. (B) Proximity ligation assay. The red dots demonstrate co-association between claudin-1 and Src proteins. The blue stain (DAPI) was used to demarcate the nucleus.

FIG. 2 shows the development of 16 as a lead compound. (A) In-silico 3D-structure of claudin-1, cavities that can be targeted by small molecules were defined using Molegro Virtual Docker. (B) Cell survival assay showing effects of three different inhibitors (I1, I2 and 16). 1-6 was most effective. (C) Effect of 16 treatment (72 hours) upon resistance to anoikis in claudin-1 overexpressing SW480 cells and claudin-1 deficient HCT116 cells. (D) Effect of 16 treatment on cell invasion in SW480claudin-1 cells. (E) Effect of inhibitor 16 on xenograft tumors. Tumors were induced for two weeks after which 16 was injected i.p (10 mg/kg). Effect on tumor growth was monitored.

FIG. 3 shows that cancer stem cell (CSC) population is markedly increased (˜10 folds) in claudin-1 overexpressing CRC cells. (A) Side population (SP) analysis; (B) Colonogenic assay using SW480claudin-1 cells and control cells; (C) qPCR for CSC markers; (D) 5FU treatment of control and claudin-1 overexpressing cells. Data is presented as mean of n=3.

FIG. 4 shows that inhibitor 16 acts as an adjuvant in co-treatment with 5FU for anti-CRC therapy. It shows synergistic effect in inhibition of cell growth. Curve fitting analysis was done using four parametric logistic regression curve.

FIG. 5 shows that co-association between Src and Claudin-1 bindings is inhibited in cells treated with I—. (A) Proximity ligation assay; indicates association between P-Src and Claudin-1 (B) Co—IP of cells treated with 16 (25, 50 μM).

FIG. 6A shows the plasma concentration-time profile of 16 after 1 mg/kg intravenous administration (mean±SD, n #5).

FIG. 6B shows pharmacokinetic parameters of 16 after 1 mg/kg intravenous administration of 16 (mean±SD, n #3).

FIG. 7 shows tissue (ng/g) concentration of 16 after 1 mg/kg intravenous administration of 16.

FIG. 8 shows the time-dependent metabolic depletion of Inhibitor-6 (16). Metabolic elimination profiles (% turnover or amount remaining vs. incubation time). Data shown as mean±S.D (n #3).

FIG. 9 shows the effect of Compound 1 and PDS-0330 treatment (72 h) upon resistance to anoikis in CLDN1 high SW480CLDN1 and SW620 cells and CLDN1 deficient HCT116 or Cldn1 inhibited SW620shRNA cells. (A) % of live and dead cells; (B) Invasion assay and (C) soft agar colony formation assay in SW480CLDN1 cells with Compound 1 (negative control).

FIG. 10A shows the Phase I metabolism of PDS-0330 in human liver microsomes. The result of the metabolic stability study was expressed as the % drug conc. remaining.

FIG. 10B shows a comparison of the Phase I metabolism of 16 and PDS-0330 in human liver microsomes. The result of the metabolic stability study was expressed as the % of the drug conc. remaining.

FIG. 11 shows the effect of 16 treatment (72 hours) upon resistance to anoikis in claudin-1 overexpressing SW480 cells and claudin-1 deficient HCT116 cells. (A) % of live and dead cells (B) image of number of cells surviving after 72 hrs (C) Effect of 1-6 treatment on cell invasion and soft agar colony formation assay in SW480claudin-1 cells.

FIG. 12 shows that normal cells or CLDN2 overexpressing cells are not affected by PDS-0030. SW480^{claudin-1}, IEC-6 (normal cells), and HCT116^{CLDN2} cells were treated with different concentrations of PDS-0330 and cell viability was determined.

FIG. 13 shows that PDS-0330 is more effective in inhibiting cell survival and p-EphA2 expression in CLDN1 overexpressing cells than dasatinib. SW480^{claudin-1} cells were treated with dasatinib or PDS-0330. Effects on (A) phosphorylation of Src and EphA2 and (B) cell survival (MTT assay) were determined. Significant changes are boxed.

FIG. 14 shows SW480^{claudin-1} over-expressing cells that were treated with Compound 1, 16 and PDS-0330 and effect on association of Cld-1 and p-Src was determined using immuno-precipitation.

FIG. 15 shows that SW480^{claudin-1} overexpressing cells were treated with Compound 1, 16 and PDS-0330 and effects on expression of p-Src, Src and c-PARP was determined.

FIG. 16 shows that DLDFUR cells (5FU resistant cells were treated with 16 and PDS-0330 and effects on expression of p-Src and c-PARP was determined.

FIG. 17 shows the biochemical and biophysical behaviors of human claudins. (A) F-SEC traces of 18 human claudins expressed in HEK-293T cells (top), and SDS-PAGE in-gel fluorescence of same F-SEC samples (bottom). (B) SEC traces of five human claudins expressed in insect cells (top), and SDS-PAGE Coomassie-stained gel showing purification of same samples (bottom). (C) FSEC-TS traces of temperature-challenged claudin. (D) Graph of data from C illustrating a Tₘ increase upon additive incubation. (E) BLI association and dissociation curves of human claudin receptor binding to known protein toxin.

FIG. 18 shows SW480claudin-1 cells xenograft tumors were grown then injected with PDS-0330 (5 and 10 mg/kg). Tumor volume and weight were measured.

FIG. 19 shows the results of orthotopic cecal transplantation of SW620 cells. (A) hepatic metastasis (arrows) B. Representative H&E-stained slide showing metastatic tumor of SW620 cells to liver (arrows).

FIG. 20 shows the results of a Claudin-1 assay. PDOX generated from liver, lung or peritoneal metastasis from different patients were tested for expression of Claudin-1 and β-actin was used as a control.

FIG. 21 shows that PDS-0330 reduces tumor size in mice. SW620 cells were injected into dorsal flanks of athymic mice to form xenograft tumors. PDS-0330 was injected (5 and 10 mg/kg) when tumors became palpable. Effect on tumor volume and size was determined in time-dependent manner.

FIG. 22 shows that PDS-0330 reduces tumor size in mice. SW620 cells were injected into dorsal flanks of athymic mice to form xenograft tumors. PDS-0330 was injected (5 and 10 mg/kg) when tumors became palpable. Effect on tumor volume and size was determined in time-dependent manner.

FIG. 23 shows that proliferation marker Ki67, apoptotic marker cleaved PARP, anti-apoptotic markers BclxL, and survivin were observed in vehicle and PDS-0330 (5 mg/kg and 10 mg/kg) treated SW620 cells xenograft tumor lysates by IHC and immunoblotting.

FIG. 24 shows the anti-tumorigenic potential of PDS-0330 in-vivo. (A) H&E (scale equals 100 μm) and Immunohistochemical analysis (scale equals 100 μm) of proliferation marker (Ki67) and apoptotic marker (cleaved PARP) were observed in SW480^{cld1} cells xenograft tumor sections. (B) Apoptotic marker cleaved PARP and anti-apoptotic markers BclxL, and survivin were observed in vehicle and PDS-0330 (5 mg/kg and 10 mg/kg) treated SW480^{cld1} cells xenograft tumor lysates by immunoblotting.

FIG. 25 shows the effect of PDS-0330 on tumor volume over time. Tumor volume was measured using calipers over time (n=5 mice per group). The formation of flank xenograft tumors in claudin-1 overexpressed SW480^{cld1} cells injected nude mice was examined between vehicle and PDS-0330 (5 mg/kg and 10 mg/kg) treatment (n=5 mice per group). For graphs, data represent mean±SD; *, P<0.05; **, P<0.01; ***, P<0.001 & ****, P<0.0001.

FIG. 26 shows representative images of tumors from the vehicle and PDS-0330 (5 mg/kg and 10 mg/kg)-treated claudin-1 overexpressed SW480^{cld1} cells injected nude mice.

FIG. 27 shows the anti-tumorigenic potential of PDS-0330 in-vivo. H&E (scale equals 100 μm) and Immunohistochemical analysis (scale equals 100 μm) of proliferation marker (Ki67) and apoptotic marker (cleaved PARP) were observed in SW620 cells xenograft tumor sections.

FIG. 28 shows that apoptotic marker cleaved PARP and anti-apoptotic markers BclxL, and survivin were observed in vehicle and PDS-0330 (5 mg/kg and 10 mg/kg) treated SW620 cells xenograft tumor lysates by immunoblotting.

FIG. 29 shows that PDS-0330 significantly inhibited tumor weight in claudin-1 overexpressed SW620 xenograft tumors.

FIG. 30 shows the development of tumors in xenograft mice treated with vehicle (negative control) and PDS-0330. The development of flank xenograft tumors in claudin-1 overexpressed SW620 cells injected nude mice was examined between vehicle and PDS-0330 (5 mg/kg and 10 mg/kg) treatment (n=5 mice per group). Representative images of tumor containing mice from the vehicle and PDS-0330 (10 mg/kg).

FIG. 31 shows that PDS-0330 inhibits tumor formation of claudin-1 overexpressed patient-derived xenograft nude mice. Representative images of tumors from the vehicle and PDS-0330 (5 mg/kg and 10 mg/kg)-treated claudin-1 overexpressed PDX implanted nude mice.

FIG. 32 shows the effect of PDS-0330 on tumor size over time. Tumor volume was measured using calipers over time (n=5 mice per group). The formation of flank xenograft tumors in claudin-1 overexpressed PDX implanted nude mice was examined between vehicle and PDS-0330 (5 mg/kg and 10 mg/kg) treatment (n=5 mice per group). For graphs, data represent mean±SD; *, P<0.05; **, P<0.01; ***, P<0.001 & ****, P<0.0001.

FIG. 33 shows that apoptotic markers BclxL, Cleaved PARP, and Survivin were observed in the vehicle, and PDS-0330 (5 mg/kg and 10 mg/kg) treated PDX tumor lysates by immunoblotting.

FIG. 34 shows that H&E (scale equals 100 μm) and Immunohistochemical analysis (scale equals 100 μm) of proliferation marker (Ki67) and apoptotic marker (cleaved PARP) were observed in PDX tumor sections.

FIG. 35 shows that PDS-0330 has no cytotoxic effect in mice by measuring blood urea nitrogen (BUN). Data are expressed as means±SEM (n=5 mice/group).

FIG. 36 shows the results of a liver toxicity assay (aspartate aminotransferase (AST) colorimetric activity assay) (concentration in blood plasma).

FIG. 37 shows the results of a liver toxicity assay (aspartate aminotransferase (AST) colorimetric activity assay) (concentration in liver).

FIG. 38 shows plasma concentration profile of PDS-0330 after 5 mg/kg post oral and 5 mg/kg intraperitoneal (IP) administration of PDS-0330 (mean±SD, n=5).

FIG. 39 shows non-compartmental pharmacokinetic parameters of PDS-0330 after 5 mg/kg post oral and 5 mg/kg IP administration of PDS-0330 (mean±SD, n=5).

FIG. 40 shows tissue concentrations of PDS-0330 following oral (5 mg/kg) or intraperitoneal (IP) (5 mg/kg) administration (mean±SD, n=5).

FIG. 41 shows tissue concentrations of PDS-0330 following oral (5 mg/kg) or intraperitoneal (IP) (5 mg/kg) administration (mean±SD, n=5).

### DETAILED DESCRIPTION

Provided herein are compounds that inhibit claudin-1, and which can treat or prevent a disease or disorder associated with claudin-1 in a subject. Any of the compounds disclosed herein can be useful in the treatment of a variety of diseases and disorders, including but not limited to hepatitis, diabetic nephropathy, ulcerative colitis, Crohn's disease, and cancer. Any diseases or disorders known to be treated, prevented or mediated by inhibition of claudin-1 can benefit from treatment with the compounds of the disclosure.

### Claudin-1 Inhibitors

There is a “focal” significance of dysregulated claudin-1 expression in integrating oncogenic signaling pathways (Src), known to promote CRC-malignancy. Despite the validation of the in vitro model of “anoikis” for studying mechanisms regulating metastasis in vivo, its functional association with cell survival, CSC and chemoresistance has not been harnessed for understanding regulation of the CRC metastasis. Without intending to be bound by theory, it is believe that emphasizing “Focal” significance of dysregulated claudin-1 expression in integrating oncolgenic singaling pathways (SRC) known to promote CRC-malignancy can beneficially expand oncogenic influence of claudin-1/Src signaling in understanding the interdependence between CSC, chemoresistance, anoikis and metastasis. Novel small molecule inhibitors, including PDS-0330, that can effectively inhibit claudin-1 dependent CRC malignancy including resistance to anoikis, cell invasion and tumor growth in vivo have been identified. Treatment with this inhibitor also disrupts claudin-1/Src association. Prior to the compounds of the disclosure, no effective anti-claudin-1 inhibitor had been identified. Considering the wide-ranging roles of dysregulated claudin-1 in cancer progression and other diseases, the compounds of the disclosure can have a wide-ranging and significant therapeutic impact.

In addition toguiding disease prevention, indetificatio of specific genetic signatures, which can serve as biomarkers for the risk of cancer aggressiveness is also provided and used to evaluate whether combinatorial claudin-1/Src profiles can be predictive of disease aggressiveness or screening for recurrence.

### Compounds of the Disclosure

The present disclosure describes compounds, compositions, and methods for the treatment of diseases and disorders capable of being modulated by claudin-1 inhibition, including cancer. The disclosure describes improved or complementary chemotherapeutics for any number of cancers including colorectal cancer.

In one embodiment the disease or disorder is cancer. In various embodiments, the cancer is breast, colorectal, pancreatic, liver, nasopharyngeal, ovarian, uterine, lung, brain, skin, gastric, ovary, esophageal, prostate, colon, stomach, or oral squamous cell cancer. In one embodiment, the cancer is colorectal cancer. In one embodiment the compositions of the present disclosure can be administered with at least one other therapeutic agent (e.g. other anti-cancer agents).

In one embodiment, the disease or disorder is hepatitis. In one embodiment, the disease or disorder is diabetic nephropathy. In one embodiment, the disease or disorder is ulcerative colitis. In one embodiment, the disease or disorder is Crohn's disease.

The compounds and compositions of the present disclosure may be formulated with any pharmaceutically acceptable carrier(s) and provided in any desired dosage form. The compositions of the present disclosure may be administered to a patient by any method. Methods of administration include but are not limited to parenterally, subcutaneously, orally, topically, pulmonarily, rectally, vaginally, intravenously, intraperitoneally, intrathecally, intracerbrally, epidurally, intramuscularly, intradermally, or intracarotidly.

The disclosure provides compounds of Formula I:

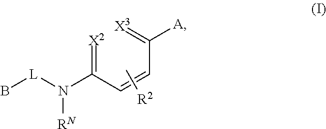

wherein
Bis C₆-C₁₀ aryl or 5-10 membered heteroaryl comprising 1-3 ring heteroatoms independently selected from O, S, and N, wherein the aryl or heteroaryl is substituted with one R¹;
X¹ is CH or N;
X² is CH or N;
X³ is CH or N;
L is

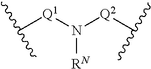

or 5-membered heteroaryl comprising 1 to 3 heteroatoms independently selected from O, S, and N;
Q¹ is a bond, C═O, or C═S;
Q² is C═O, C═S, or SO₂;
A is C₃-C₁₀ cycloalkyl, 3-10 membered heterocycloalkyl comprising 1-3 ring heteroatoms independently selected from O, S, and N, C₆-C₁₀ aryl, or 5-10 membered heteroaryl comprising 1-3 ring heteroatoms independently selected from O, S, and N, wherein the cycloalkyl, heterocycloalkyl, aryl, and heteroaryl are optionally substituted with 1-3 R³;
R¹, R², and R³ are each independently H, halo, OH, CN, C₁₋₆ alkyl, C₁₋₆ haloalkyl, C₁₋₆ hydroxyalkyl, C₁₋₆ alkoxy, or C₁₋₆ haloalkoxy; and
each R^{N} is independently H or C₁₋₆ alkyl, or two R^{N} together with the atoms to which they are attached form a 5-6 membered heterocycloalkyl ring further comprising 0 or 1 additional ring heteroatoms selected from O, S, and N.

In some cases, B is C₆-C₁₀ aryl substituted with one R¹. In some cases, B is

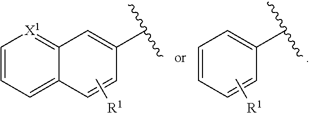

In some cases, B is

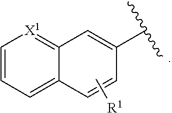

In some cases, B is

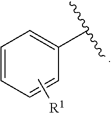

In some cases, B is phenyl or naphthyl substituted with one R¹. In some cases, B is phenyl substituted with one R¹. In some cases, B is naphthyl substituted with one R¹. In some cases, B is 5-10 membered heteroaryl comprising 1-3 ring heteroatoms independently selected from O, S, and N, substituted with one R¹. In some cases, B is quinolinyl substituted with one R¹. In some cases, X¹ is CH. In some cases, X¹ is N.

In some cases, the compound has the structure of Formula Ia or Ib:

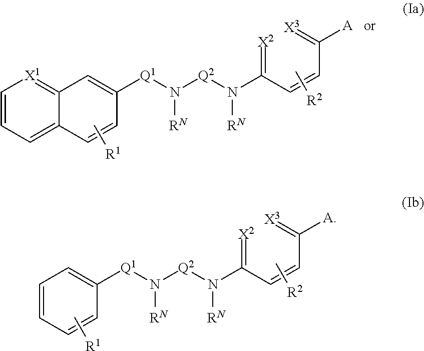

In some cases, the compound has the structure of Formula Ia:

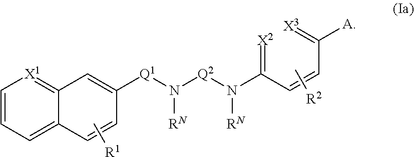

In some cases, the compound has the structure of

### Formula Ib:

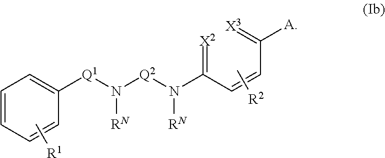

In some cases, X² is CH. In some cases, X² is N. In some cases, X³ is CH. In some cases, X³ is N.

In some cases, L is

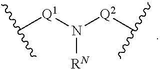

In some cases, Q¹ is a bond or C═O. In some cases, Q¹ is a bond. In some cases, Q¹ is C═O. In some cases, Q¹ is C═S. In some cases, Q² is C═O or C═S. In some cases, Q² is C═O. In some cases, Q² is C═S. In some cases, Q² is SO₂. In some cases, L is

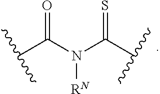

In some cases, L is

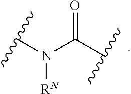

In some cases, L is

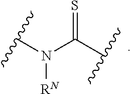

In some cases, L is 5-membered heteroaryl comprising 1 to 3 heteroatoms independently selected from O, S, and N. In some cases, L is imidazole, triazole, thiadiazole, or oxadiazole.

In some cases, A is C₃-C₁₀ cycloalkyl. In some cases, A is 3-10 membered heterocycloalkyl comprising 1-3 ring heteroatoms independently selected from O, S, and N. In some cases, A is C₆-C₁₀ aryl. In some cases, A is 5-10 membered heteroaryl comprising 1-3 ring heteroatoms independently selected from O, S, and N optionally substituted with 1-3 R³. In some cases, A is 5-6 membered heteroaryl comprising 1-3 ring heteroatoms independently selected from O, S, and N optionally substituted with 1-3 R³. In some cases, A is 8-10 membered heteroaryl comprising 1-3 ring heteroatoms independently selected from O, S, and N optionally substituted with 1-3 R³. In some cases, A is unsubstituted. In some cases, A is benzimidazole or benzoxazole. In some cases, A is benzimidazole. In some cases, A is benzoxazole. In some cases, A is

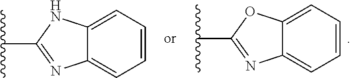 [SMILES: *c1nc2ccccc2[nH]1 ; *c1nc2ccccc2o1]

In some cases, A is

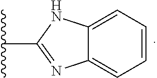 [SMILES: *c1nc2ccccc2[nH]1]

In some cases, A is

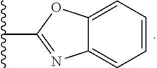 [SMILES: *c1nc2ccccc2o1]

In some cases, R¹, R², and R³ are each independently H, halo, OH, CN, C₁₋₆ alkyl, C₁₋₆-haloalkyl, C₁₋₆ hydroxyalkyl, C₁₋₆ alkoxy, or C₁₋₆ haloalkoxy. In some cases, R¹ is H or C₁₋₆ alkoxy. In some cases, R¹ is H. In some cases, R¹ is halo. In some cases, R¹ is OH. In some cases, R¹ is CN. In some cases, R¹ is C₁₋₆ alkyl. In some cases, R¹ is C₁₋₆ haloalkyl. In some cases, R¹ C₁₋₆ hydroxyalkyl. In some cases, R¹ is C₁₋₆ alkoxy. In some cases, R¹ is methoxy. In some cases, R¹ is C₁₋₆ haloalkoxy. In some cases, R² is H. In some cases, R² is halo. In some cases, R² is OH. In some cases, R² is CN. In some cases, R² is C₁₋₆ alkyl. In some cases, R² is C₁₋₆ haloalkyl. In some cases, R² C₁₋₆ hydroxyalkyl. In some cases, R² is C₁₋₆ alkoxy. In some cases, R² is C₁₋₆ haloalkoxy. In some cases, R³ is H. In some cases, R³ is halo. In some cases, R³ is OH. In some cases, R³ is CN. In some cases, R³ is C₁₋₆ alkyl. In some cases, R³ is C₁₋₆ haloalkyl. In some cases, R³ C₁₋₆ hydroxyalkyl. In some cases, R³ is C₁₋₆-alkoxy. In some cases, R³ is C₁₋₆ haloalkoxy.

In some cases, at least one R^{N} is H. In some cases, each R^{N} is H. In some cases, at least one R^{N} is C₁₋₆ alkyl. In some cases, each R^{N} is C₁₋₆ alkyl. In some cases, two R^{N} together with the atoms to which they are attached form a 5-6 membered heterocycloalkyl ring further comprising 0 or 1 additional ring heteroatoms selected from O, S, and N.

Specific compounds contemplated include those listed in Table 1, or a pharmaceutically acceptable salt thereof:

TABLE 1

| Compound | |
|---|---|
| No. | Structure |
| 1 (also called 19A) | 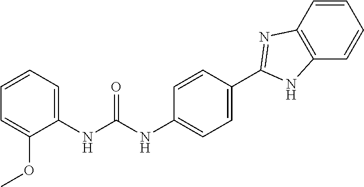 [SMILES: COc1ccccc1NC(=O)Nc1ccc(-c2nc3ccccc3[nH]2)cc1] |
| 2 (also called 19B) | 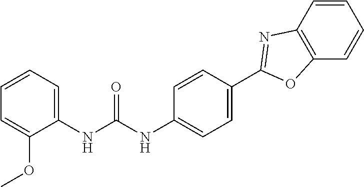 [SMILES: COc1ccccc1NC(=O)Nc1ccc(-c2nc3ccccc3o2)cc1] |
| 3 (also called 20A) | 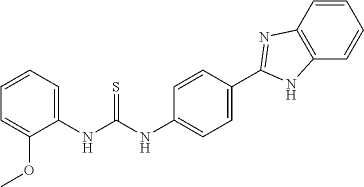 [SMILES: COc1ccccc1NC(=S)Nc1ccc(-c2nc3ccccc3[nH]2)cc1] |
| 4 (also called 20B) | 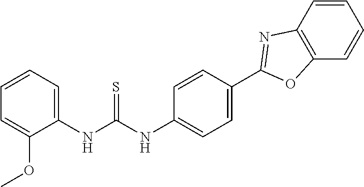 [SMILES: COc1ccccc1NC(=S)Nc1ccc(-c2nc3ccccc3o2)cc1] |
| 5 (also called 22A) | 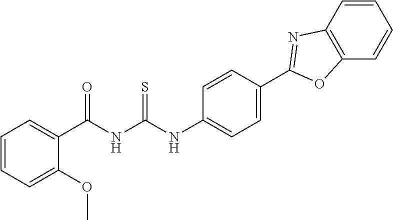 [SMILES: COc1ccccc1C(=O)NC(=S)Nc1ccc(-c2nc3ccccc3o2)cc1] |
| 6 (also called 22B) | 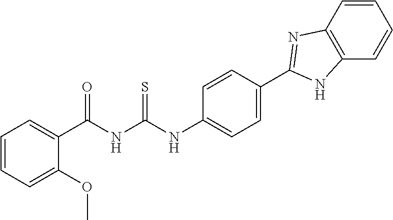 [SMILES: COc1ccccc1C(=O)NC(=S)Nc1ccc(-c2nc3ccccc3[nH]2)cc1] |
| 7 (also claled 23A or PDS-0330) | 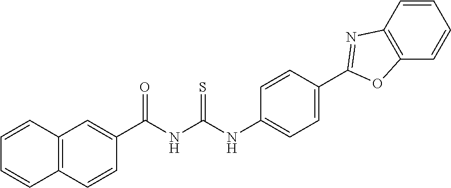 [SMILES: O=C(NC(=S)Nc1ccc(-c2nc3ccccc3o2)cc1)c1ccc2ccccc2c1] |
| 8 (also called 23B) | 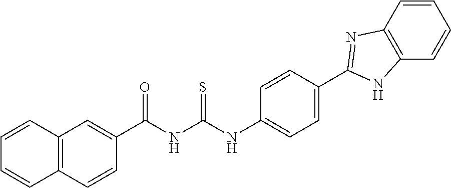 [SMILES: O=C(NC(=S)Nc1ccc(-c2nc3ccccc3[nH]2)cc1)c1ccc2ccccc2c1] |
| 9 (also called 16 or 25B) | 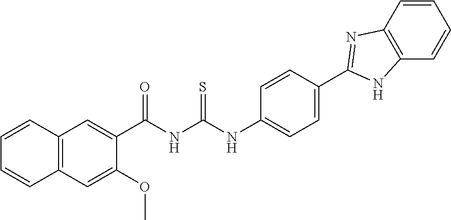 [SMILES: COc1cc2ccccc2cc1C(=O)NC(=S)Nc1ccc(-c2nc3ccccc3[nH]2)cc1] |
| 10 (also called 25C) | 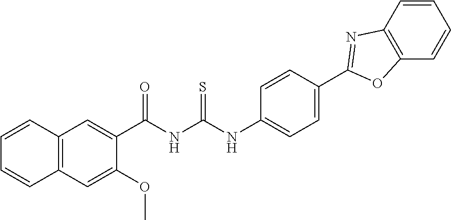 [SMILES: COc1cc2ccccc2cc1C(=O)NC(=S)Nc1ccc(-c2nc3ccccc3o2)cc1] |

In some cases, the compound is compound 7 (also called PDS-0330) or compound 9 (also called 16). In some cases, the compound is compound 7 (also called PDS-0330).

The compounds disclosed herein can be in the form of a pharmaceutically acceptable salt. As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al. describe pharmaceutically acceptable salts in detail in J. Pharmaceutical Sciences, 1977, 66, 1-19, which is incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, trifluoroacetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, glutamate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts of compounds containing a carboxylic acid or other acidic functional group can be prepared by reacting with a suitable base. Such salts include, but are not limited to, alkali metal, alkaline earth metal, aluminum salts, ammonium, N⁺(C₁₋₄alkyl)₄ salts, and salts of organic bases such as trimethylamine, triethylamine, morpholine, pyridine, piperidine, picoline, dicyclohexylamine, N, N′-dibenzylethylenediamine, 2-hydroxyethylamine, bis-(2-hydroxyethyl) amine, tri-(2-hydroxyethyl) amine, procaine, dibenzylpiperidine, dehydroabietylamine, N, N′-bisdehydroabietylamine, glucamine, N-methylglucamine, collidine, quinine, quinoline, and basic amino acids such as lysine and arginine. This disclosure also envisions the quaternization of any basic nitrogen-containing groups of the compounds disclosed herein. Water or oil-soluble or dispersible products may be obtained by such quaternization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

### Definitions

As used herein, the term “alkyl” refers to straight chained and branched saturated hydrocarbon groups containing one to six carbon atoms. The term Cn means the alkyl group has “n” carbon atoms. For example, C₆ alkyl refers to an alkyl group that has 6 carbon atoms. C₁-C₆ alkyl refers to an alkyl group having a number of carbon atoms encompassing the entire range (e.g., 1 to 6 carbon atoms), as well as all subgroups (e.g., 1-6, 1-5, 3-6, 1, 2, 3, 4, 5, and 6 carbon atoms). Nonlimiting examples of alkyl groups include, methyl, ethyl, n-propyl, isopropyl, n-butyl, sec-butyl (2-methylpropyl), and t-butyl (1,1-dimethylethyl). Unless otherwise indicated, an alkyl group can be an unsubstituted alkyl group or a substituted alkyl group.

As used herein, the term “alkoxy” refers to a “—O-alkyl” group. The alkoxy group can be unsubstituted or substituted.

As used herein, the term “haloalkyl” refers to an alkyl group substituted with one or more halogen substituents. For example, C₁-C₆haloalkyl refers to a C₁-C₆ alkyl group substituted with one or more halogen atoms, e.g., 1, 2, 3, 4, 5, or 6 halogen atoms. Non-limiting examples of haloalkyl groups include fluoromethyl, difluoromethyl, trifluoromethyl, chloromethyl, dichloromethyl, and trichloromethyl groups. Similarly, haloalkoxy refers to an alkoxy group substituted with one or more halogen atoms e.g., 1, 2, 3, 4, 5, or 6 halogen atoms.

As used herein, the term “hydroxyalkyl” refers to an alkyl group substituted with one or more OH substituents. For example, C₁-C₆hydroxyalkyl refers to a C₁-C₆ alkyl group substituted with one or more OH groups, e.g., 1, 2, or 3 OH groups.

As used herein, the term “halo” or “halogen” refers to fluorine, chlorine, bromine, or iodine.

As used herein, the term “isothiocyanate” refers to a —N═C═S group.

As used herein, the term “cycloalkyl” refers to an aliphatic cyclic hydrocarbon group containing three to ten carbon atoms (e.g., 3, 4, 5, 6, 7, 8, 9, or 10 carbon atoms). The term Cn means the cycloalkyl group has “n” carbon atoms. For example, C₅ cycloalkyl refers to a cycloalkyl group that has 5 carbon atoms in the ring. C₃-C₁₀ cycloalkyl refers to cycloalkyl groups having a number of carbon atoms encompassing the entire range (e.g., 3 to 10 carbon atoms), as well as all subgroups (e.g., 3-4, 3-5, 3-6, 3-7, 3-8, 3-9, 3-10, 4-5, 4-6, 4-7, 4-8, 4-9, 4-10, 5-6, 5-7, 5-8, 5-9, 5-10, 6-7, 6-8, 7-8, 6-9, 6-10, 6, 7, 8, 9, and 10 carbon atoms). Nonlimiting examples of cycloalkyl groups include cyclopropyl, cyclobutyl, cyclopentyl, cyclohexyl, cycloheptyl, and cyclooctyl. Unless otherwise indicated, a cycloalkyl group can be an unsubstituted cycloalkyl group or a substituted cycloalkyl group.

As used herein, the term “heterocycloalkyl” is defined similarly as cycloalkyl, except the ring contains one to three heteroatoms independently selected from oxygen, nitrogen, and sulfur. In particular, the term “heterocycloalkyl” refers to a ring containing a total of three to twelve atoms (e.g., three to seven, or five to ten), of which 1, 2, 3 or three of those atoms are heteroatoms independently selected from the group consisting of oxygen, nitrogen, and sulfur, and the remaining atoms in the ring are carbon atoms. Nonlimiting examples of heterocycloalkyl groups include piperdine, pyrazolidine, tetrahydrofuran, tetrahydropyran, dihydrofuran, morpholine, and the like.

As used herein, the term “aryl” refers to a monocyclic aromatic group, such as phenyl. Unless otherwise indicated, an aryl group can be unsubstituted or substituted with one or more, and in particular one to four groups independently selected from, for example, halo, alkyl, alkenyl, OCF₃, NO₂, CN, NC, OH, alkoxy, amino, CO₂H, CO₂alkyl, aryl, and heteroaryl. Aryl groups can be isolated (e.g., phenyl) or fused to another aryl group (e.g., naphthyl, anthracenyl), a cycloalkyl group (e.g. tetraydronaphthyl), a heterocycloalkyl group, and/or a heteroaryl group. Exemplary aryl groups include, but are not limited to, phenyl, chlorophenyl, methylphenyl, methoxyphenyl, trifluoromethylphenyl, nitrophenyl, 2,4-methoxychlorophenyl, and the like.

As used herein, the term “heteroaryl” refers to an aromatic ring having 5 to 10 total ring atoms, and containing one to four heteroatoms selected from nitrogen, oxygen, and sulfur atom in the aromatic ring. Unless otherwise indicated, a heteroaryl group can be unsubstituted or substituted with one or more, and in particular one to four, substituents. Non-limiting examples of heteroaryl groups include, but are not limited to, benzimidazolyl, benzoxazolyl, quinolinyl, thienyl, furyl, pyridyl, pyrrolyl, oxazolyl, oxadiazolyl, triazinyl, triazolyl, thiadiazolyl, isothiazolyl, isoxazolyl, indolyl, imidazolyl, pyrazinyl, pyrimidinyl, thiazolyl, and thiadiazolyl.

As used herein, the term “substituted,” when used to modify a chemical functional group, refers to the replacement of at least one hydrogen radical on the functional group with a substituent. Substituents can include, but are not limited to, alkyl, cycloalkyl, alkenyl, cycloalkenyl, alkynyl, heterocycloalkyl, aryl, heteroaryl, hydroxyl, oxy, alkoxy, heteroalkoxy, ester, thioester, carboxy, cyano, nitro, amino, amido, acetamide, and halo (e.g., fluoro, chloro, bromo, or iodo). When a chemical functional group includes more than one substituent, the substituents can be bound to the same carbon atom or to two or more different carbon atoms.

As used herein, the phrase “optionally substituted” means unsubstituted (e.g., substituted with a H) or substituted. As used herein, the term “substituted” means that a hydrogen atom is removed and replaced by a substituent. It is understood that substitution at a given atom is limited by valency. The use of a substituent (radical) prefix name such as alkyl without the modifier “optionally substituted” or “substituted” is understood to mean that the particular substituent is unsubstituted.

As used herein, the term “therapeutically effective amount” means an amount of a compound or combination of therapeutically active compounds (e.g., a claludin-1 inhibitor) that ameliorates, attenuates or eliminates one or more symptoms of a particular disease or condition (e.g., cancer), or prevents or delays the onset of one of more symptoms of a particular disease or condition.

As used herein, the terms “patient” and “subject” may be used interchangeably and mean animals, such as dogs, cats, cows, horses, and sheep (e.g., non-human animals) and humans. Particular patients or subjects are mammals (e.g., humans). The terms patient and subject include males and females.

As used herein, the term “pharmaceutically acceptable” means that the referenced substance, such as a compound of the present disclosure, or a formulation containing the compound, or a particular excipient, are safe and suitable for administration to a patient or subject. The term “pharmaceutically acceptable excipient” refers to a medium that does not interfere with the effectiveness of the biological activity of the active ingredient(s) and is not toxic to the host to which it is administered.

As used herein, the term “excipient” means any pharmaceutically acceptable additive, carrier, diluent, adjuvant, or other ingredient, other than the active pharmaceutical ingredient (API).

### Synthesis of Compounds of the Disclosure

The compounds disclosed herein can be prepared in a variety of ways using commercially available starting materials, compounds known in the literature, or from readily prepared intermediates, by employing standard synthetic methods and procedures either known to those skilled in the art, or in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5th edition, John Wiley & Sons: New York, 2001; and Greene, T. W., Wuts, P. G. M., Protective Groups in Organic Synthesis, 3^{rd} edition, John Wiley & Sons: New York, 1999, are useful and recognized reference textbooks of organic synthesis known to those in the art. For example, the compounds disclosed herein can be synthesized by solid phase synthesis techniques including those described in Merrifield, J. Am. Chem. Soc. 1963; 85:2149; Davis et al., Biochem. Intl. 1985; 10:394-414; Larsen et al., J. Am. Chem. Soc. 1993; 115:6247; Smith et al., J. Peptide Protein Res. 1994; 44:183; O'Donnell et al., J. Am. Chem. Soc. 1996; 118:6070; Stewart and Young, Solid Phase Peptide Synthesis, Freeman (1969); Finn et al., The Proteins, 3rd ed., vol. 2, pp. 105-253 (1976); and Erickson et al., The Proteins, 3rd ed., vol. 2, pp. 257-527 (1976). The following descriptions of synthetic methods are designed to illustrate, but not to limit, general procedures for the preparation of compounds of the present disclosure.

The synthetic processes disclosed herein can tolerate a wide variety of functional groups; therefore, various substituted starting materials can be used. The processes generally provide the desired final compound at or near the end of the overall process, although it may be desirable in certain instances to further convert the compound to a pharmaceutically acceptable salt, ester or prodrug thereof.

### Pharmaceutical Formulations, Dosing, and Routes of Administration

Further provided are pharmaceutical formulations comprising a compound as described herein (e.g., compounds of Formula I, la, lb, or compounds of Table 1, or pharmaceutically acceptable salts of the compounds) and a pharmaceutically acceptable excipient.

The compounds described herein can be administered to a subject in a therapeutically effective amount (e.g., in an amount sufficient to prevent or relieve the symptoms of a disorder associated with aberrant claudin-1 activity). The compounds can be administered alone or as part of a pharmaceutically acceptable composition or formulation. In addition, the compounds can be administered all at once, multiple times, or delivered substantially uniformly over a period of time. It is also noted that the dose of the compound can be varied over time.

A particular administration regimen for a particular subject will depend, in part, upon the compound, the amount of compound administered, the route of administration, and the cause and extent of any side effects. The amount of compound administered to a subject (e.g., a mammal, such as a human) in accordance with the disclosure should be sufficient to effect the desired response over a reasonable time frame. Dosage typically depends upon the route, timing, and frequency of administration. Accordingly, the clinician titers the dosage and modifies the route of administration to obtain the optimal therapeutic effect, and conventional range-finding techniques are known to those of ordinary skill in the art.

Purely by way of illustration, the method comprises administering, e.g., from about 0.1 mg/kg up to about 100 mg/kg of compound or more, depending on the factors mentioned above. In other embodiments, the dosage ranges from 1 mg/kg up to about 100 mg/kg; or 5 mg/kg up to about 100 mg/kg; or 10 mg/kg up to about 100 mg/kg. Some conditions require prolonged treatment, which may or may not entail administering lower doses of compound over multiple administrations. If desired, a dose of the compound is administered as two, three, four, five, six or more sub-doses administered separately at appropriate intervals throughout the day, optionally, in unit dosage forms. The treatment period will depend on the particular condition and type of pain, and may last one day to several months.

Suitable methods of administering a physiologically-acceptable composition, such as a pharmaceutical composition comprising the compounds disclosed herein (e.g., compounds of Formula I, la, Ib, or compounds of Table 1, or pharmaceutically acceptable salts of the compounds), are well known in the art. Although more than one route can be used to administer a compound, a particular route can provide a more immediate and more effective reaction than another route. Depending on the circumstances, a pharmaceutical composition comprising the compound is applied or instilled into body cavities, absorbed through the skin or mucous membranes, ingested, inhaled, and/or introduced into circulation. For example, in certain circumstances, it will be desirable to deliver a pharmaceutical composition comprising the agent orally, through injection by intravenous, intraperitoneal, intracerebral (intra-parenchymal), intracerebroventricular, intramuscular, intra-ocular, intraarterial, intraportal, intralesional, intramedullary, intrathecal, intraventricular, transdermal, subcutaneous, intraperitoneal, intranasal, enteral, topical, sublingual, urethral, vaginal, or rectal means, by sustained release systems, or by implantation devices. If desired, the compound is administered regionally via intrathecal administration, intracerebral (intra-parenchymal) administration, intracerebroventricular administration, or intraarterial or intravenous administration feeding the region of interest. Alternatively, the composition is administered locally via implantation of a membrane, sponge, or another appropriate material onto which the desired compound has been absorbed or encapsulated. Where an implantation device is used, the device is, in one aspect, implanted into any suitable tissue or organ, and delivery of the desired compound is, for example, via diffusion, timed-release bolus, or continuous administration.

To facilitate administration, the compound is, in various aspects, formulated into a physiologically-acceptable composition comprising a carrier (e.g., vehicle, adjuvant, or diluent). The particular carrier employed is limited only by chemico-physical considerations, such as solubility and lack of reactivity with the compound, and by the route of administration. Physiologically-acceptable carriers are well known in the art. Illustrative pharmaceutical forms suitable for injectable use include sterile aqueous solutions or dispersions and sterile powders for the extemporaneous preparation of sterile injectable solutions or dispersions (for example, see U.S. Pat. No. 5,466,468). Injectable formulations are further described in, e.g., Pharmaceutics and Pharmacy Practice, J. B. Lippincott Co., Philadelphia. Pa., Banker and Chalmers, eds., pages 238-250 (1982), and ASHP Handbook on Injectable Drugs, Toissel, 4th ed., pages 622-630 (1986)). A pharmaceutical composition comprising the compound is, in one aspect, placed within containers, along with packaging material that provides instructions regarding the use of such pharmaceutical compositions. Generally, such instructions include a tangible expression describing the reagent concentration, as well as, in certain embodiments, relative amounts of excipient ingredients or diluents (e.g., water, saline or PBS) that may be necessary to reconstitute the pharmaceutical composition.

Compositions suitable for parenteral injection may comprise physiologically acceptable sterile aqueous or nonaqueous solutions, dispersions, suspensions, or emulsions, and sterile powders for reconstitution into sterile injectable solutions or dispersions. Examples of suitable aqueous and nonaqueous carriers, diluents, solvents, or vehicles include water, ethanol, polyols (propylene glycol, polyethylene glycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants.

These compositions may also contain adjuvants such as preserving, wetting, emulsifying, and dispersing agents. Microorganism contamination can be prevented by adding various antibacterial and antifungal agents, for example, parabens, chlorobutanol, phenol, sorbic acid, and the like. It may also be desirable to include isotonic agents, for example, sugars, sodium chloride, and the like. Prolonged absorption of injectable pharmaceutical compositions can be brought about by the use of agents delaying absorption, for example, aluminum monostearate and gelatin.

Solid dosage forms for oral administration include capsules, tablets, powders, and granules. In such solid dosage forms, the active compound is admixed with at least one inert customary excipient (or carrier) such as sodium citrate or dicalcium phosphate or (a) fillers or extenders, as for example, starches, lactose, sucrose, mannitol, and silicic acid; (b) binders, as for example, carboxymethylcellulose, alginates, gelatin, polyvinylpyrrolidone, sucrose, and acacia; (c) humectants, as for example, glycerol; (d) disintegrating agents, as for example, agar-agar, calcium carbonate, potato or tapioca starch, alginic acid, certain complex silicates, and sodium carbonate; (a) solution retarders, as for example, paraffin; (f) absorption accelerators, as for example, quaternary ammonium compounds; (g) wetting agents, as for example, cetyl alcohol and glycerol monostearate; (h) adsorbents, as for example, kaolin and bentonite; and (i) lubricants, as for example, talc, calcium stearate, magnesium stearate, solid polyethylene glycols, sodium lauryl sulfate, or mixtures thereof. In the case of capsules, and tablets, the dosage forms may also comprise buffering agents. Solid compositions of a similar type may also be used as fillers in soft and hard filled gelatin capsules using such excipients as lactose or milk sugar, as well as high molecular weight polyethylene glycols, and the like.

Solid dosage forms such as tablets, dragees, capsules, pills, and granules can be prepared with coatings and shells, such as enteric coatings and others well known in the art. The solid dosage forms may also contain opacifying agents. Further, the solid dosage forms may be embedding compositions, such that they release the active compound or compounds in a certain part of the intestinal tract in a delayed manner. Examples of embedding compositions that can be used are polymeric substances and waxes. The active compound can also be in micro-encapsulated form, optionally with one or more excipients.

Liquid dosage forms for oral administration include pharmaceutically acceptable emulsions, solutions, suspensions, syrups, and elixirs. In addition to the active compounds, the liquid dosage form may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propylene glycol, 1,3-butylene glycol, dimethylformamide, oils, in particular, cottonseed oil, groundnut oil, corn germ oil, olive oil, castor oil, and sesame seed oil, glycerol, tetrahydrofurfuryl alcohol, polyethylene glycols and fatty acid esters of sorbitan, or mixtures of these substances, and the like.

Besides such inert diluents, the composition can also include adjuvants, such as wetting agents, emulsifying and suspending agents, sweetening, flavoring, and perfuming agents. Suspensions, in addition to the active compound, may contain suspending agents, as for example, ethoxylated isostearyl alcohols, polyoxyethylene sorbitol and sorbitan esters, microcrystalline cellulose, aluminum metahydroxide, bentonite, agar-agar, and tragacanth, or mixtures of these substances, and the like.

Compositions for rectal administration are preferably suppositories, which can be prepared by mixing the compounds of the disclosure with suitable non-irritating excipients or carriers such as cocoa butter, polyethylene glycol or a suppository wax, which are solid at ordinary room temperature, but liquid at body temperature, and therefore, melt in the rectum or vaginal cavity and release the active component.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration.

The frequency of dosing will depend on the pharmacokinetic parameters of the agents and the routes of administration. The optimal pharmaceutical formulation will be determined by one of skill in the art depending on the route of administration and the desired dosage. See, for example, Remington's Pharmaceutical Sciences, 18th Ed. (1990) Mack Publishing Co., Easton, PA, pages 1435-1712, incorporated herein by reference. Such formulations may influence the physical state, stability, rate of in vivo release and rate of in vivo clearance of the administered agents. Depending on the route of administration, a suitable dose may be calculated according to body weight, body surface areas or organ size. Further refinement of the calculations necessary to determine the appropriate treatment dose is routinely made by those of ordinary skill in the art without undue experimentation, especially in light of the dosage information and assays disclosed herein, as well as the pharmacokinetic data observed in animals or human clinical trials.

The precise dosage to be employed depends upon several factors including the host, whether in veterinary medicine or human medicine, the nature and severity of the condition, e.g., disease or disorder, being treated, the mode of administration and the particular active substance employed. The compounds may be administered by any conventional route, in particular enterally, and, in one aspect, orally in the form of tablets or capsules. Administered compounds can be in the free form or pharmaceutically acceptable salt form as appropriate, for use as a pharmaceutical, particularly for use in the prophylactic or curative treatment of a disease of interest. These measures will slow the rate of progress of the disease state and assist the body in reversing the process direction in a natural manner.

It will be appreciated that the pharmaceutical compositions and treatment methods of the disclosure are useful in fields of human medicine and veterinary medicine. Thus the subject to be treated is in one aspect a mammal. In another aspect, the mammal is a human.

In jurisdictions that forbid the patenting of methods that are practiced on the human body, the meaning of “administering” of a composition to a human subject shall be restricted to prescribing a controlled substance that a human subject will self-administer by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

### Methods of Use

The compounds described herein (e.g., the compounds of Formula I, la, Ib, and compounds of Table 1, or pharmaceutically acceptable salts of the compounds) can inhibit claudin-1. In various embodiments, the compounds are claudin-1 modulators, e.g., the compounds change, inhibit, or prevent one or more of claudin-1's biological activities.

The compounds disclosed herein are particularly advantageous for the treatment of diseases or disorders caused by aberrant expression or activity of claudin-1. The incidence and/or intensity of diseases or disorders associated with aberrant expression or activity of claudin-1 is reduced.

Increased expression and/or activity of claudin-1 includes overexpression or hyperactivity of any component of a claudin-1 pathway, including e.g., association with Src/EphA2. Overexpression and/or hyperactivity of claudin-1 is known to cause many adverse conditions. These include, for example, hepatitis, diabetic nephropathy, ulcerative colitis, Crohn's disease, and cancer. Cancer includes but is not limited to breast, colorectal, pancreatic, liver, nasopharyngeal, ovarian, uterine, lung, brain, skin, gastric, ovary, esophageal, prostate, colon, stomach, or oral squamous cell cancer. In some cases, the cancer is colorectal cancer.

Claudin-1 inhibitors can be used for cancer prevention and/or treatment.

Compounds of Formula I, la, Ib, and compounds of Table 1, and pharmaceutically acceptable salts of the compounds display high selectivity for growth inhibition and/or induction of apoptosis in cancer cells, e.g., in colorectal cancer cells. The compounds disclosed herein selectively inhibit the growth and/or induce apoptosis in cancer cells that overexpress claudin-1 without affecting normal cells.

Provided herein is a method of modulating claudin-1 in a cell, comprising contacting the cell with a compound or a composition as disclosed herein (e.g., the compounds of Formula I, la, Ib, or compounds of Table 1, or pharmaceutically acceptable salts of the compounds) in an amount sufficient to modulate the claudin-1 pathway. The contacting of the cell can occur in vitro or in vivo. In some cases, contacting of the cell occurs in vitro. In other cases, contacting of the cell occurs in vivo. Therefore, the disclosure includes administering one or more of a compound described herein to a subject, such as a human, in need thereof. In some embodiments, the subject suffers from a disease or disorder associated with aberrant activity of claudin-1. These include, for example, hepatitis, diabetic nephropathy, ulcerative colitis, Crohn's disease, and cancer. Cancer includes but is not limited to breast, colorectal, pancreatic, liver, nasopharyngeal, ovarian, uterine, lung, brain, skin, gastric, ovary, esophageal, prostate, colon, stomach, or oral squamous cell cancer. In some cases, the cancer is colorectal cancer.

The disclosed methods utilize compounds that inhibit claudin-1, for treating, e.g., cancer. Methods for assessing the usefulness of a compound for treating cancer are known to those of skill in the art. For example, compounds may be assessed using models of cancer, including cells (such as colorectal cancer cells), animal models (such as mouse xenograph or other cancer models), or in human subjects having, e.g., colorectal cancer.

The compounds described herein can be used to decrease or prevent cancer in subjects with e.g., colorectal cancer. The subject can be human. In a particular example, a compound or mixture is administered orally, such as by mixing with distilled water. In another example, a compound or mixture is administered intravenously, such as in saline or distilled water. In some examples, treatment with a test compound (e.g., a compound of the disclosure) may be a single dose or repeated doses. The test compound may be administered about every 6 hours, about every 12 hours, about every 24 hours (daily), about every 48 hours, about every 72 hours, or about weekly. Treatment with repeated doses may continue for a period of time, for example for about 1 week to 12 months, such as about 1 week to about 6 months, or about 2 weeks to about 3 months, or about 1 to 2 months. Administration of a compound may also continue indefinitely. Doses of test compound are from about 0.1 mg/kg to about 400 mg/kg, such as about 1 mg/kg to about 300 mg/kg, about 2 mg/kg to 200 mg/kg, about 10 mg/kg to about 100 mg/kg, about 20 mg/kg to about 75 mg/kg, or about 25 mg/kg to about 50 mg/kg.

It will be understood that the methods and compositions described herein for treating cancer, comprising administering a compound that inhibits claudin-1, are applicable to methods of treating other diseases related to claudin-1 activity, such as those described above. The methods for assessing the effectiveness of compounds for treating such diseases in cells, appropriate animal models, or affected subjects are known to one of skill in the art.

Uses of the compounds disclosed herein in the preparation of a medicament for treating diseases or disorders related to claudin-1 activity, e.g., cancer, also are provided herein.

The disclosure herein will be understood more readily by reference to the following examples, below.

### Examples

The following examples are provided for illustration and are not intended to limit the scope of the disclosure.

CRC progression, especially the influence of claudin-1 dysregulation in this progression, was studied using mice genetically manipulated for overexpressing (Villin-Claudin-1 transgenic) and interbred with APCmin mice (APCmin/Cld-1 mice). Colorectal cancer mouse models were studied using colonoscopy. Colonoscopy not only increases feasibility of the longitudinal analysis of CRC progression, it also limits animal sacrifices. A 3D-organoid culture from mouse models of colon cancer, chemoresistant cells, proximity ligation assay, and CRC patient samples with advanced and chemoresistant disease were used to explore the role of claudin-1 as prognostic biomarker for disease severity.

### Claudin-1 Expression Promotes EMT and Colon Cancer Metastasis:

Claudin-1 expression is highly upregulated in colon cancer, where highest claudin-1 expression was detected in metastatic samples and CRC cell lines (5-7, 14, 15). To examine the causal role of claudin-1 in CRC progression, claudin-1 was expressed in a non-metastatic CRC cell line, SW480 cells, to achieve claudin-1 expression comparable to SW620 cells, the highly metastatic and high claudin-1 expressing cells derived from the same genetic background. In vivo analysis using these cells in a mouse model of hepatic metastasis [intra-splenic injections] demonstrated a positive association between claudin-1 expression and hepatic metastasis of CRC cells (Published in JCI (7)). These data support a positive causal association between claudin-1 expression and CRC progression/metastasis.

Claudin-1 Associates with Src in a Multiprotein Complex.

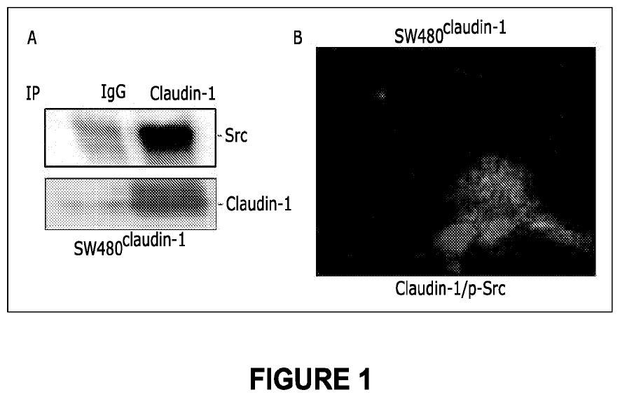

To metastasize, tumor cells need to detach from the parent tumor mass and travel to the distant organs before colonizing. This is why “resistance to anoikis” is considered a key regulator of cancer malignancy (invasion and metastasis) including in colon cancer. There is a causal role of claudin-1 in promoting resistance to anoikis in CRC cells, and a positive correlation between claudin-1 expression, resistance to anoikis and Src phosphorylation along with phosphorylation of focal adhesion proteins (Akt and paxillin) (9). It was observed that Claudin-1 associates with Src in a complex (FIG. 1A, (9)). These results were further confirmed using proximity ligation assay (PLA) (FIG. 1B). It was further observed that the c-terminal-motif of claudin-1 is critical for its partnering with Src and to regulate resistance to anoikis. In this regard, inhibiting Src phosphorylation using a pharmacological inhibitor or overexpression of claudin-1 mutant protein (lacking PDZ motif or C-terminal domain) decreased claudin-1-dependent resistance to anoikis (9). Overall, this suggests that claudin-1 associates with Src, through its c-terminal, to promote resistance to anoikis/metastasis in CRC cells.

### Compound Library Design

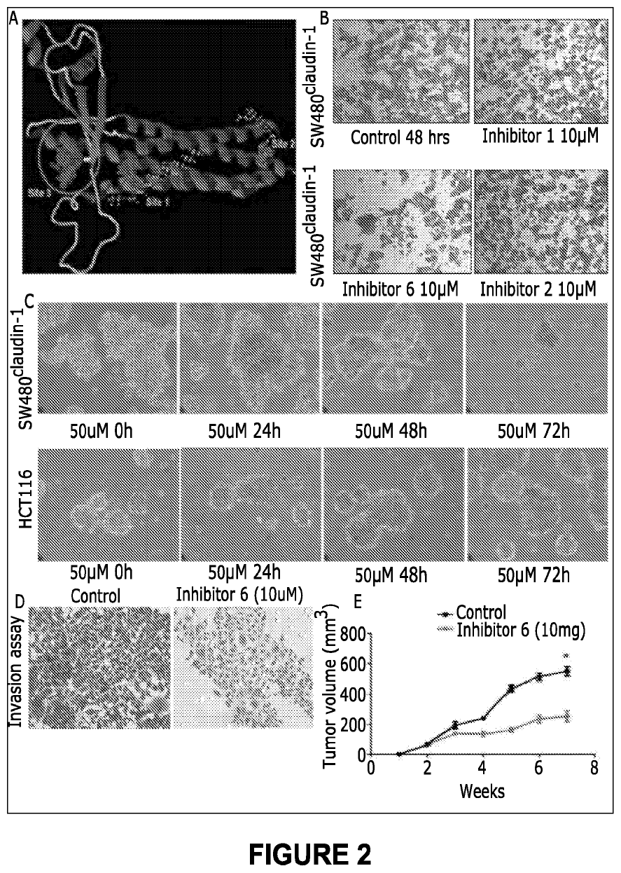

The crystal structure of claudin-1 is unknown. Therefore, an in-silico protein folding program, YASARA, was used to identify an effective claudin-1 inhibitor. The resultant 3D-structure was then modeled using Molegro Virtual Docker (MVD) software program. These in-silico analyses identified putative cavities in the claudin-1 protein that can be targeted by small molecule inhibitors. Next, a 100K library of small molecules was screened in silico using the MVD. The top 10 inhibitors that bound to the Site #3 (targeting near c-terminal domain; the most accessible pocket; FIG. 2A) with the best binding energies were selected as hits and used for further testing. Screening of these inhibitors named Inihibitor-1 (1-1) to Inhibitor-10 (I-10) was done using SW480claudin-1, SW620 and control cells. Claudin-1 deficient HCT116 cells (WT-APC) or SW620shRNAcld-1 were used as negative controls. Resistance to anoikis and cell invasion was used as biological end-points in testing these inhibitors. An extensive analysis identified Inhibitor 6 (16) as the most effective small molecule against claudin-1 mediated increases in resistance to anoikis, and cell invasion (FIG. 2B-D). 16 has the following structure:

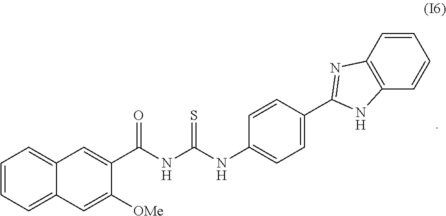 [SMILES: COc1cc2ccccc2cc1C(=O)NC(=S)Nc1ccc(-c2nc3ccccc3[nH]2)cc1]

HCT116 cells remained unaffected from the effects of 16, supporting specificity of this inhibitor for claudin-1 mediated CRC-progression. Most importantly, growth of xenograft tumors from SW480claudin-1 cells was significantly inhibited on treatment with 16, however we did not observe any toxic effects on these mice (FIG. 2E). This suggested an important role of 16 on claudin-1 mediated tumorigenesis and possibility of its use as a lead compound for further medicinal chemistry optimization.

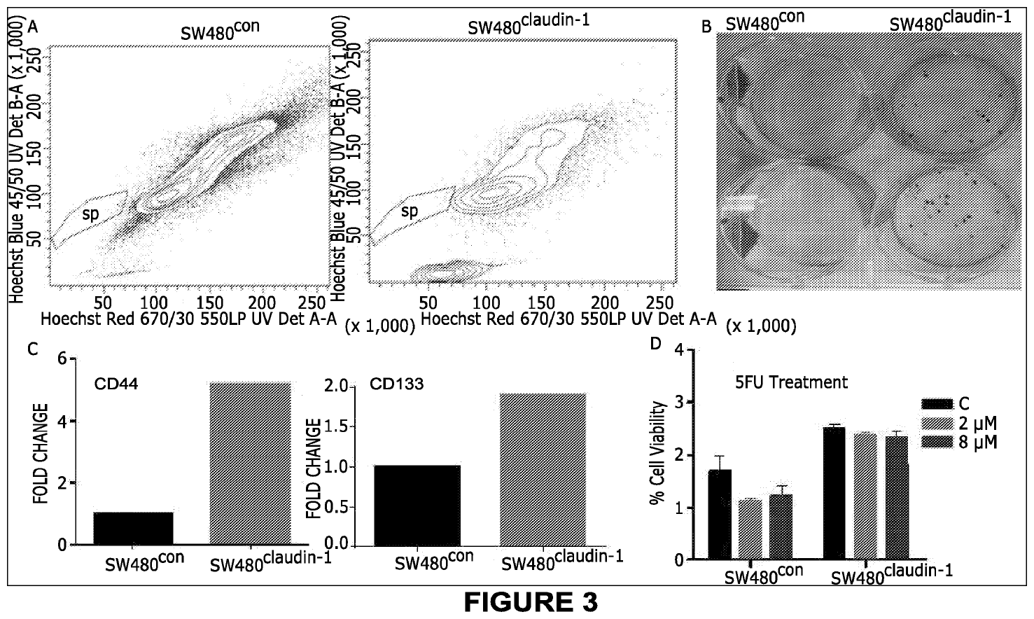

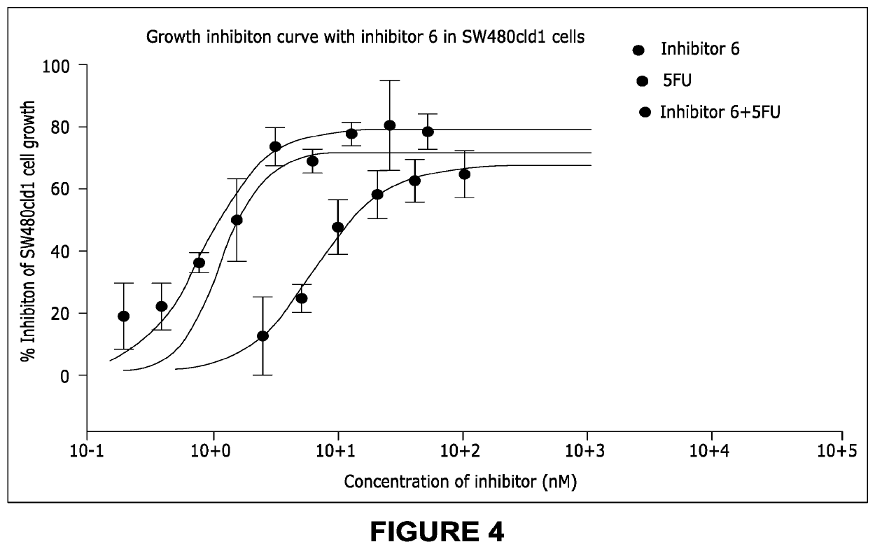

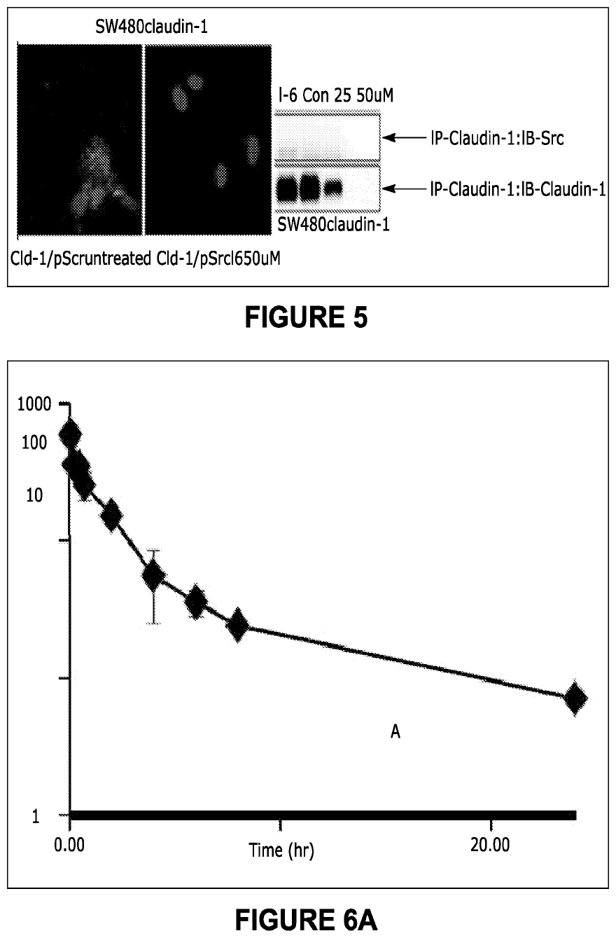

Claudin-1 overexpressing cells demonstrate chemoresistance and increase in cancer stem cell (CSC) population. The CSC cells contribute to the generation of metastasis (16-19), and key CSC characteristics are highly relevant to metastasis, including invasive motility, and, resistance to apoptosis (20,21). Notably, claudin-1 overexpressing CRC cells are not only highly metastatic (vs. control cells), but have increased CSC population as seen by increased side population, increased colony formation and increased expression of the CD44 and CD133 (FIG.-3A-C). Furthermore, treatment with 5FU demonstrated increased resistance due to claudin-1 overexpression (FIG. 3D) suggesting positive correlation between claudin-1 expression and CSC marker expression. Considering that chemoresistance is central to the CRC progression and metastasis, we further tested if adjuvant use of 16 with 5FU can have significant bearing upon these traits. Colon cancer cells were subjected to exogenous administration of 16 alone, or in combination with the 5-FU, the conventional anti-CRC drug. Results were highly encouraging as we found significant synergy in anti-cancer effects of 5-FU and 16 in claudin-1 overexpressing cells (FIG. 4). To further determine the mechanistic details the effect of 16 on association of claudin-1 and Src, SW480claudin-1 cells were treated with 16 or the vehicle and resultant cell lysate was subjected to Proximity ligation assay (PLA) or Co—IP. The claudin-1/Src complexing was markedly suppressed in 16 treated SW480claudin-1 cells (vs untreated cells; FIG. 5).

### Pilot Studies for 16 Pharmacokinetics and Tissue Distribution In Vivo:

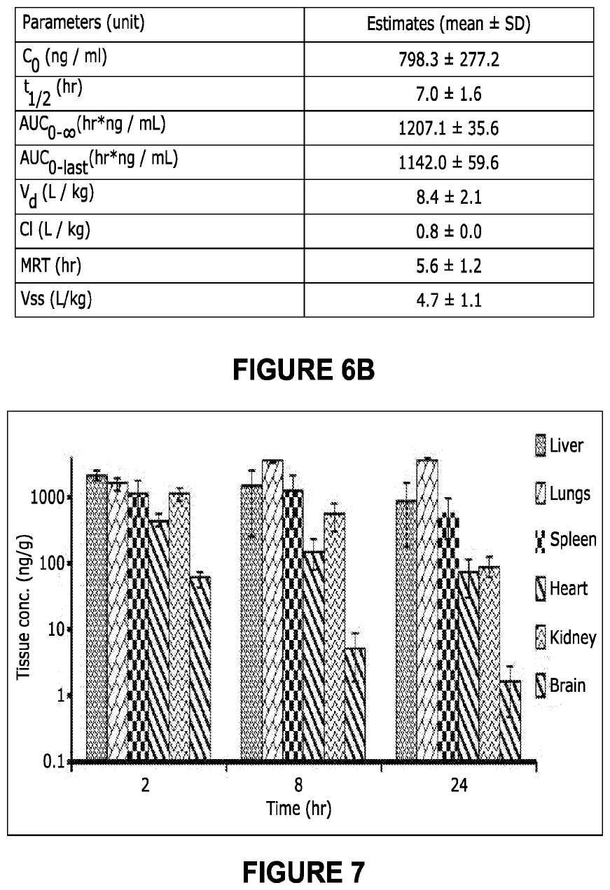

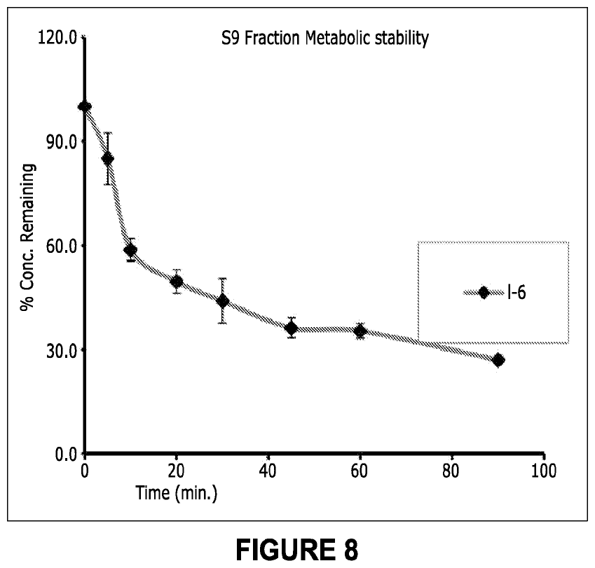

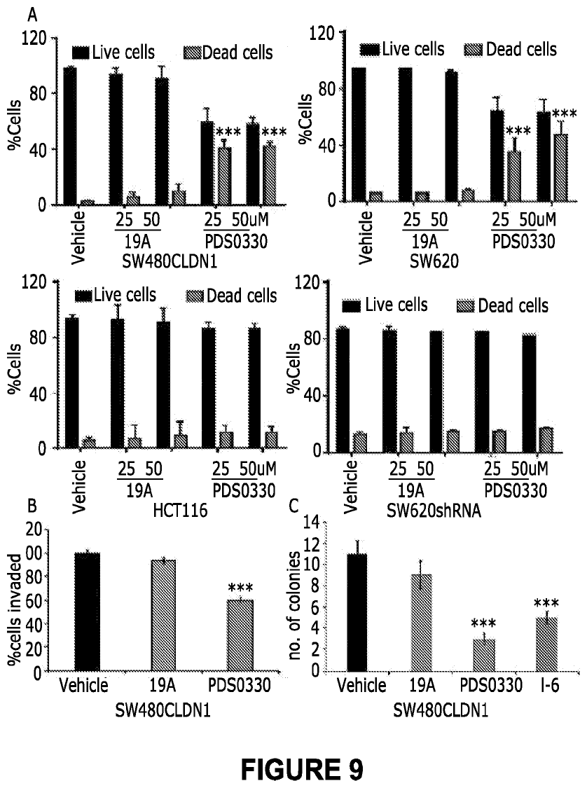

To determine the in vivo characteristics of this drug, a pharmacokinetic and tissue distribution study on 16 was performed. Following a single i.v. dose of 16 (1 mg/kg) in mice (C57B/16; n=5), serial blood samples were obtained at 0.08, 0.25, 0.75, 2, 4, 6, 8 and 24 hours after the injection and plasma samples were analyzed for 16 concentrations. Pharmacokinetic parameter values were obtained from the plasma concentration-time data using standard non-compartmental methods (WinNonlin, PharSight Corp). The plasma concentration vs. time profile after intravenous administration of 16 in mice is shown in FIG. 6A. Following the i.v. administration, peak 16 concentrations of 798 ng/ml were achieved, with an AUCO-∞ 1207 hr×ng/ml. 16 was detectable up to 24 hrs, with an elimination t1/2 of 7.0 hrs. The clearance and volume of distribution was found 0.8 L/hr/kg and 8.4 L/kg, respectively. The PK parameters of 16 in mice are summarized in FIG. 6B. To assess the tissue distribution of 16, mice were sacrificed at 2, 8 and 24 hours after the i.v. administration of the drug. Measurable concentrations of 16 were detected in all studied tissues at 2, 8, and 24 h. The tissue concentrations are shown in Table 2 and FIG. 7, which shows that 16 accumulated in tissues, with higher concentrations in the lung and liver tissue (principal sites for CRC metastasis). In addition, the data shows that the tissue to plasma concentration ratio (Kp) is ≥1 for all tissues except brain, indicating good distribution of 16 in tissues. The Kp ratios for 16 were observed in the descending order of lungs>liver>spleen>kidney>heart>>brain. Higher concentrations in different tissues, however, suggest that further toxicological studies are required to investigate the relationship between drug concentration and effect, both therapeutically and toxically, after chronic dosing. Notably, there was no change in mouse behavior or weight 24 hours after a single i.v. injection of 16 (data not shown). Together, this pilot study suggests that 16 achieves plasma and tissue concentrations above the IC50 identified in in vitro studies. Next, metabolic stability studies of 16 was evaluated using mouse liver S9 fractions for the in vitro assessment of phase I and II metabolism. 16 was found to be rapidly degraded with only 30% of the drug remaining after 60 minutes (FIG. 8). Testosterone, 7-HC and diclofenac were used as positive controls, ensuring proper incubation conditions were maintained. Primary metabolites identified included acetylation and hydroxylation with subsequent glucuronidation. The results of these in vitro drug metabolism studies guided the rational development of 16 structural analogs.

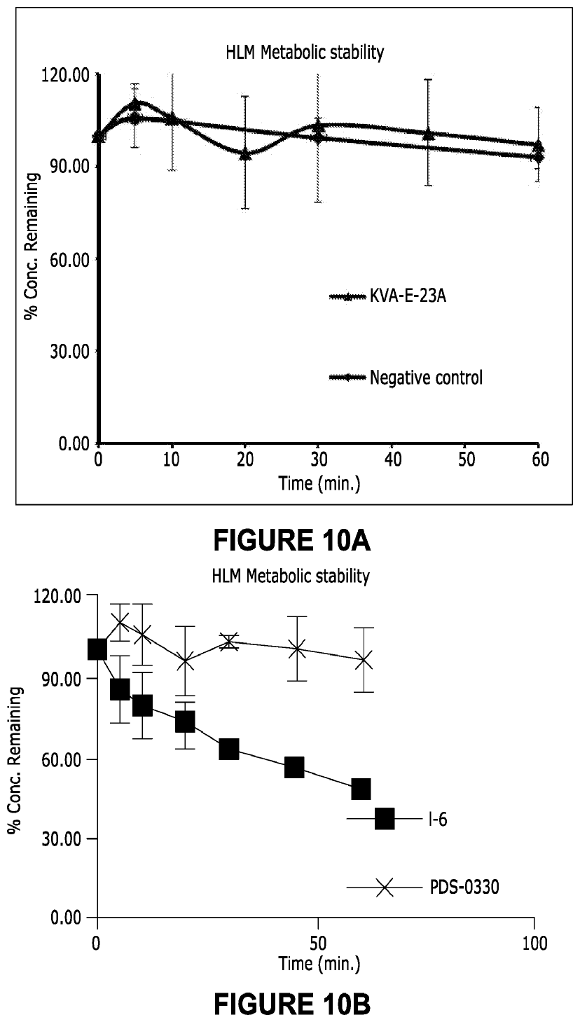

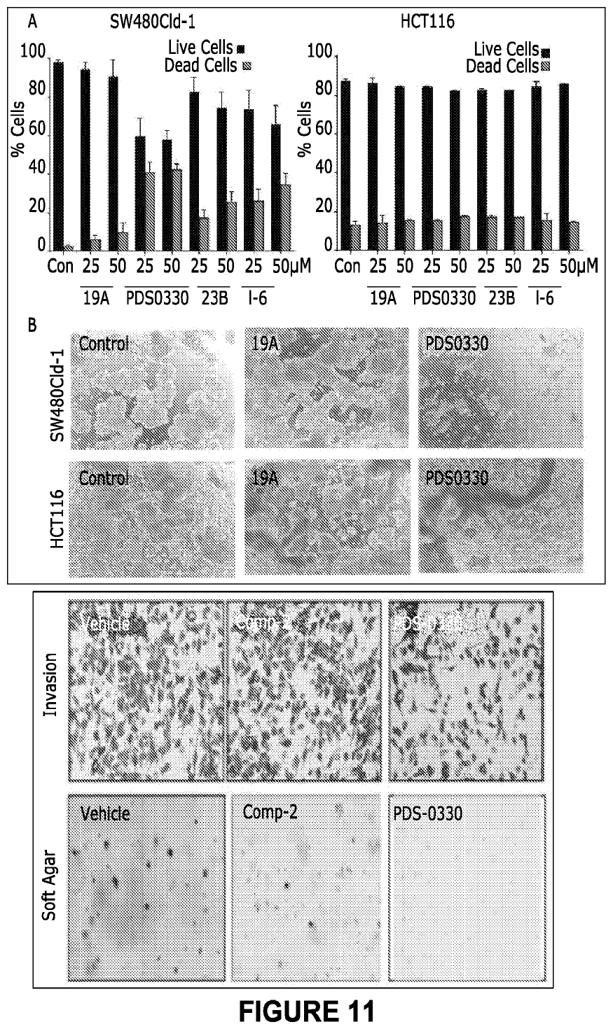

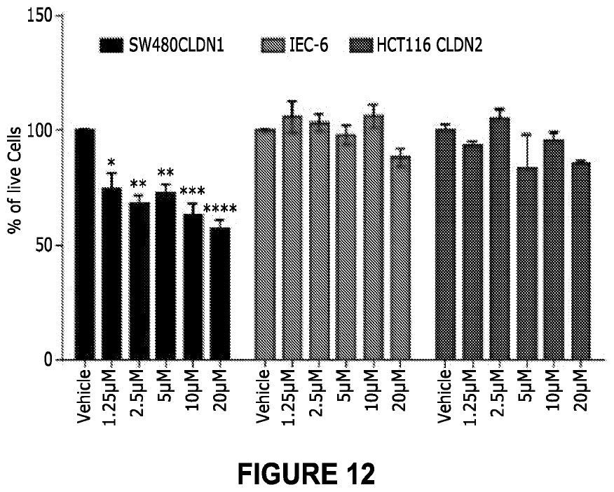

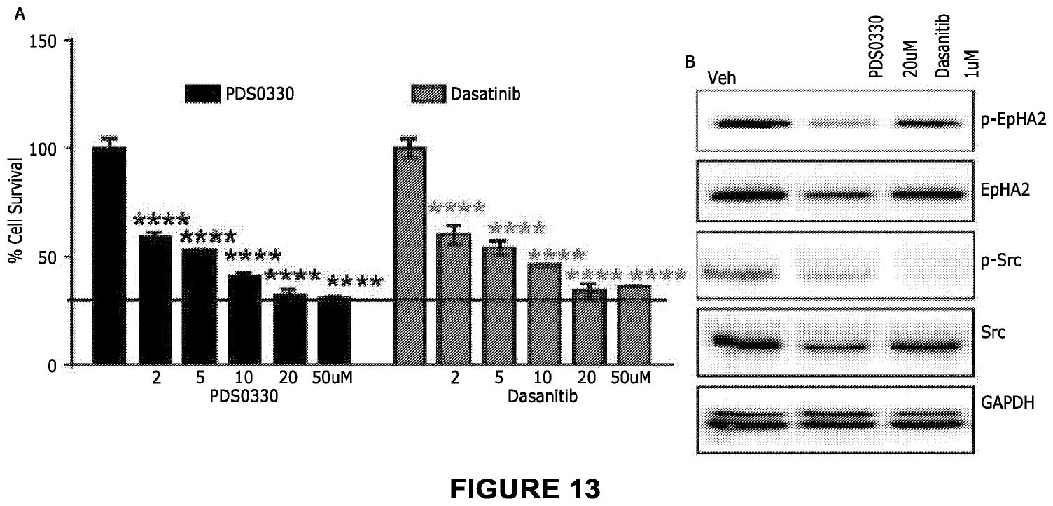

In order to progress the molecule into advanced in vitro and in vivo studies, a more stable compound than 16 was needed. Therefore, a lead optimization campaign was undertaken in order to improve the potency, selectivity and pharmacokinetic properties of 16. To this end, a library of molecules was synthesized in order to develop an initial SAR evaluation. The first compounds tested eliminated the acylated (thio) urea as well as the napthyl group and evaluated just the urea/phenyl compounds (Compounds 1-6). The methoxy group of the naphthalene ring system was needed when the benzimidazole group was present (16 vs. Compound 8). Compound 7 (also referred to as PDS-0330), that does not have the methoxy and the benzimidazole was substituted for a benzoxazole and the activity was ˜3-fold improved (PDS-0330, 8.4 μM vs. 16, 28.4 μM) (FIG. 9). Incubations of PDS-0330 with human liver microsomes showed that the compound was metabolically stable over 60 minutes as compared to 16, presumably due to the removal of the methoxy group (FIG. 8,10). The result of metabolic stability was expressed as the percentage of drug remaining at each time. These analogs were tested for resistance to anoikis and, again, the most potent compound was PDS-0330, while Compound 1 was used as negative control with not much change and Compound 8 was another analog having similar activity to 16 (FIG. 11). With repeated experiments PDS-0330 was identified as the most potent effector. Similar to previous studies with 16, PDS-0330 significantly inhibited resistance to anoikis, soft agar and invasion in SW480claudin-1 cells (FIG. 11). To further determine the effect of PDS-0330 on p-Src expression and association, cells were treated with Compound 1, 16 and PDS-0330. Expression and association with p-Src was unaffected with Compound 1 (negative control). The specificity of PDS-0330 for CLDN1 was further confirmed by treating SW480^{CLDN1}, HCT116^{CLDN2} (overexpressing another tight junction protein) or normal IEC-6 cells with PDS-0330 (FIG. 12). While SW480^{CLDN1}, as expected, showed significant increase in the percentage of dead cells, HCT116^{CLDN2} cells and IEC-6 cells were not affected by this inhibitor.

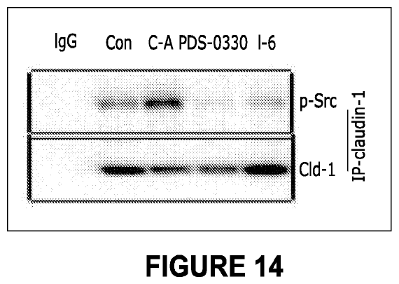

The effect of PDS-0330 in comparison to the known FDA approved inhibitor of Src, Dasatinib, was also tested. PDS-0330 significantly decreased percent cell survival more than dasatinib, and also inhibited p-EphA2 expression along with p-Src while the dasatinib effect was limited to inhibition of p-Src expression (FIG. 13).

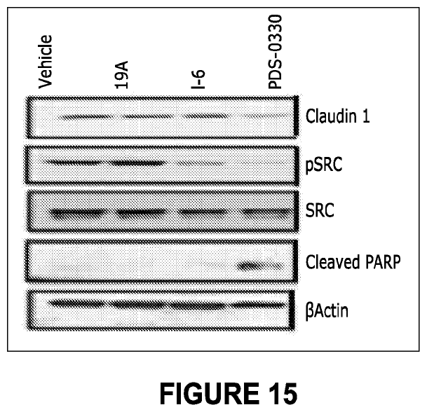

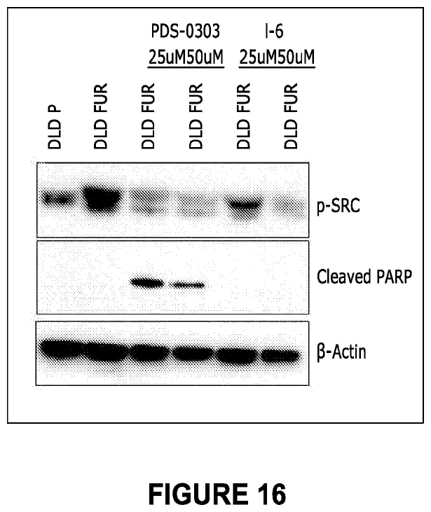

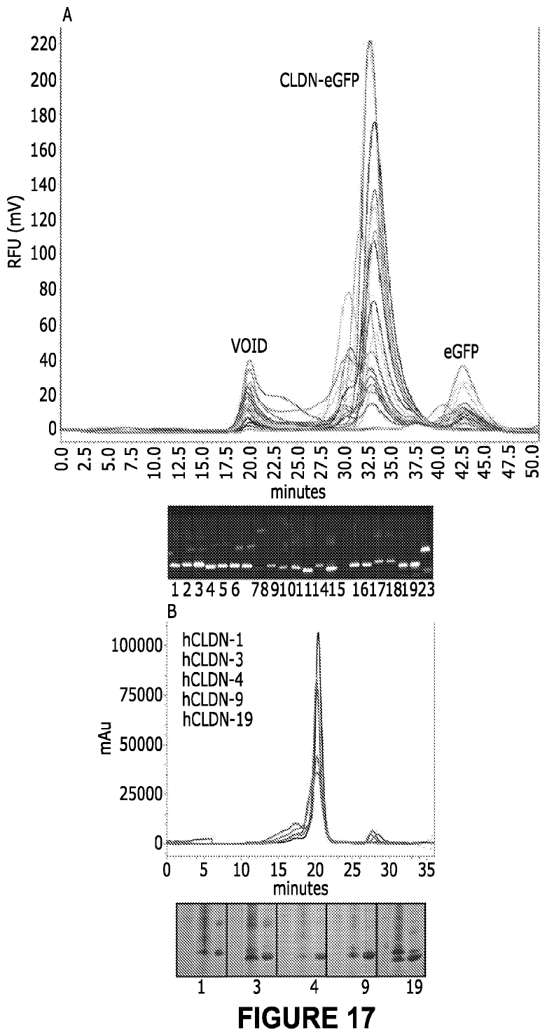

While 16 reduced p-Src expression and association with p-Src and increased apoptosis (c-PARP), the most potent effects were observed with PDS-0330 (FIG. 14, 15). Furthermore, DLD-1 chemoresistant cells were treated with PDS-0330 and I-6. A decrease in p-Src expression and increase in apoptosis (c-PARP) was observed, and the effects were more potent than I-6 alone (FIG. 16). Based on the in vitro activity and metabolic stability, PDS-0330, and any potential new analogs will be evaluated in animal models to determine the pharmacokinetic properties and biodistribution as described in preliminary data.

### Synthesis of the Compounds of the Disclosure

An example protocol for synthesizing compounds disclosed herein is shown in Scheme 1. The synthesis is accomplished using ammonium thiocyanate reaction with the acyl chloride and an appropriately substituted aniline yielding 2. (27) This procedure allows for gram-scale synthesis, if needed. The triazole analogs, 3, can be accessed using the product, 2, formed above by reacting with hydrazine (N₂H₄) as shown in Scheme 2. (28) The thiadiazole, 5, and oxadiazole, 6, can be synthesized using a common intermediate, 10 as shown in Scheme 3. (29) To this end, the N-phenylhydrazinecarbothioamide, 10, can be reacted with the carboxylic acid, 4, which will cyclize to give the target compound, 5. Amine intermediate 10 and the acid 4, can also be reacted using the carbodiimide coupling reagent which will give the oxadiazole target compound, 6. Lastly, the 1,2,4-oxadiazole, 9, can be obtained via a two-step procedure as outlined in Scheme 4. The hydroxybenimidic acid, 7 (either commercially available or synthesized in one-step), can be reacted with ethyl carbonochloridate which cyclizes and then followed by reaction with POCl₃ which provides the chlorooxadiazole, 8. (1) Finally, the aniline can be reacted to displace chloro which provides the target, 9.

While each of Schemes 1˜4 shows the synthesis of compounds having a 10-membered aryl or heteroaryl in the “B” position (as defined in Formula I), it will be understood that appropriate starting materials can be chosen to produce compounds having any of the C₆-C₁₀ aryl or 5-10 membered heteroaryl comprising 1-3 ring heteroatoms independently selected from O, S, and N, wherein the aryl or heteroaryl is substituted with one R¹, as defined in Formula I. A skilled artisan will understand which starting materials to select and/or how to modify the reaction conditions in order to synthesize the desired compound of Formula I.

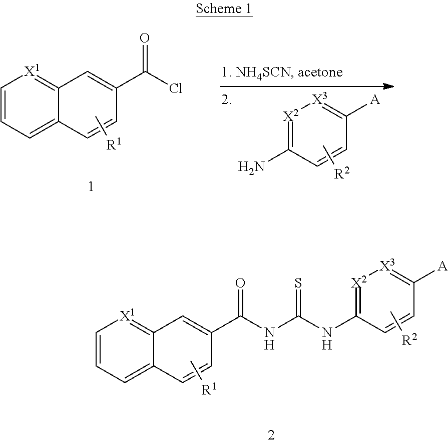

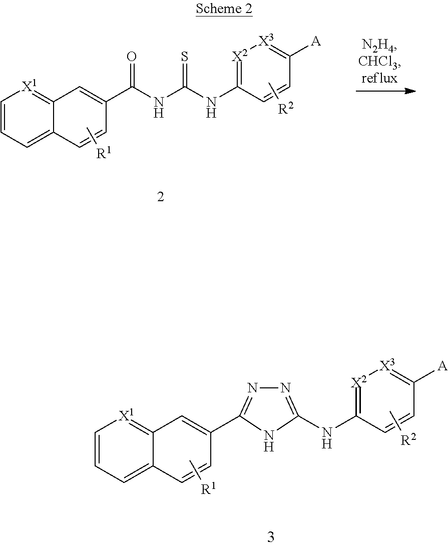

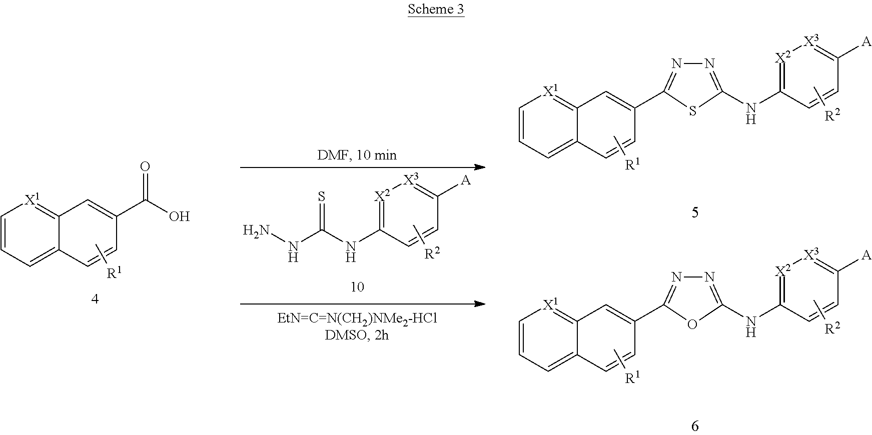

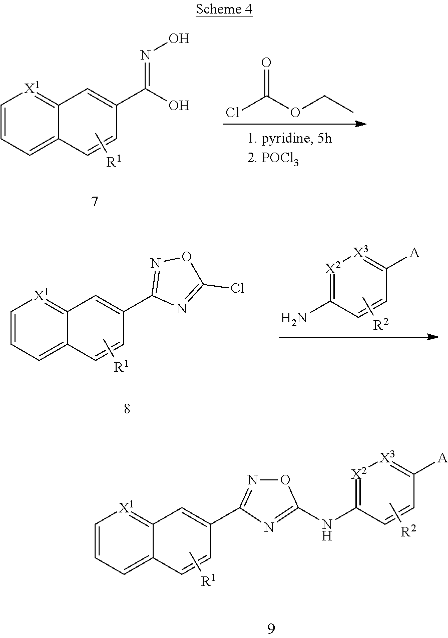

Compounds of the disclosure were synthesized, and their molecular masses were determined as shown in Table 2 below.

TABLE 2

| Compound No. | MW |
|---|---|
| 1 | 358.4 |
| 2 | 359.4 |
| 3 | 374.5 |
| 4 | 375.4 |
| 5 | 403.5 |
| 6 | 402.5 |
| 7 | 423.5 |
| 8 | 422.5 |
| 9 | 452.5 |
| 10 | 453.5 |

### Evaluation of In Vitro and In Vivo Pharmacokinetic Properties.

Initial In Vitro Drug Metabolism and Pharmacokinetics (DMPK) assessment. Three important characteristics of drug molecule are aqueous solubility, plasma free fraction, and microsomal stability. For aqueous solubility, the compounds were evaluated in two assays that used: 1. Fasted State Simulated Intestinal Fluid (FaSSIF) and 2. simulated gastric fluid (SGF). (30) Utilizing these experiments allows for insight into the potential absorption behavior after oral administration at two distinct pH environments (FaSSIF, PH ˜6.8; SGF, PH ˜1.2). Compounds disclosed herein advantageously possess solubility >10 μM.

Analytical methodology is used to quantitate and evaluate active drugs for suitable ADME properties including pH dependent stability studies, metabolic stability, plasma protein binding (PPB), absorption and efflux analysis and metabolite identification. In addition, compounds determined to have optimal ADME properties are subjected to pharmacokinetic (PK) analysis after formulation for IP (or oral) and intravenous administration to include: 1) plasma half-life 2) bioavailability analysis 3) drug clearance and 4) tissue biodistribution. The metabolism, pharmacokinetic and bioavailability data were analyzed for determination of optimal oral formulation and dose in preparation for pre-IND enabling toxicology studies.

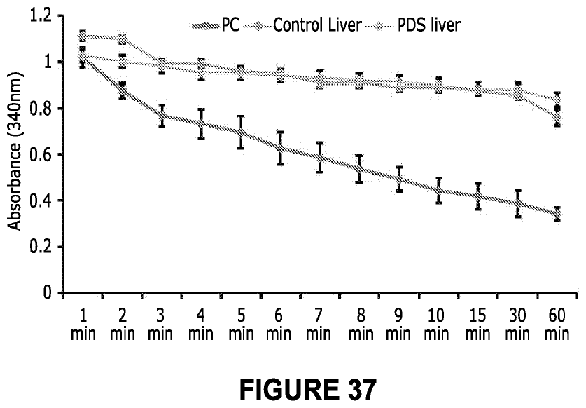

Bioanalytical LC-MS/MS Assay development: Protocols for detecting the novel compound by LC-MS/MS and for the quantitation of each active chemotype were developed as previously described for other small molecule compounds (31-34). Blood (CB) and plasma concentrations (PC) were determined by LC-MS/MS and the blood-to-plasma partition ratio (CB/CP) calculated (FIGS. 6A, 6B, 7, and 38).

pH dependent stability studies: Compounds are incubated with simulated gastrointestinal fluid and tris buffer (50 mM, pH 7.4) at 37° C. on a shaking water bath each in triplicate. Samples (100 μL) will collected at 0, 15, 30, 60 and 120 min. All samples will be analyzed by LC-MS/MS to estimate the percentage loss with respect to zero-minute time point. Calculation for stability study data can be presented as the percentage of parent compound remaining at each time.

Metabolic stability and reaction phenotyping: The in vitro metabolic stability of studies was determined using mice and human liver microsomes and S9 fractions (XenoTech, LLC, Lenexa, KS, USA) following standard protocols (33, 35, 36). Microsome stability assays were used to determine in vitro half-life (t1/2) and intrinsic clearance (CLINT). Studies weree performed utilizing 60-min time courses. FIGS. 9 and 11 show preliminary data for the lead compounds. Drug concentrations were determined using LC-MS/MS. If the HLM assays display >30% CYP-mediated metabolism, then reaction phenotyping is performed using the panel of CYP1A2, 2C9, 2C19, 2D6 and 3A4. UGTs are analyzed in Corning® Supersomes™ if incubation of HLMs with UDPGA also results in metabolite formation.

Plasma Protein Binding (PPB) study: Plasma protein binding (PPB) is determined for each selected novel compound as described using an equilibrium dialysis kit (37-39). Briefly, following plasma incubation with two concentrations of novel compound (1 μM and 10 UM) buffer is added the plate sealed and incubated at 37° C. on an orbital shaker at 100 rpm for 5 h. Ice-cold acetonitrile is added, and matrix-matched samples are vortexed and analyzed by LC-MS/MS. The PPB is calculated using standard equations.

Absorption and efflux analysis: Intestinal permeability is assessed using Caco-2 cells (ATCC), a human colon carcinoma cell line grown in differentiated monolayers on transwells. (40). Directional transport assays are performed as described previously (41). The permeability coefficient (Papp) is calculated for each tested compound to predict whether the compound is orally available.

Metabolite identification: For metabolite identification study, mass shift analyses are performed to identify structural changes in the mass fragments. The parent drug MS and MS/MS spectrum is used as a template model for structural identification and the fragmentation pattern of the parent drug from the product-ion spectrum is used to deduce the structures of the unknown metabolites (36).

Pharmacokinetics and biodistribution in mice: PK and biodistribution studies were designed to confirm that the novel compounds possess properties and stability necessary to achieve preferential retention and drug concentrations deemed effective based on in vitro studies. Eight-week-old healthy Balb/c mice (male and female) mice were utilized for the pharmacokinetic and biodistribution studies. In this section, up to two different administration routes (oral or IP) were evaluated for each selected drug product. The specified drug and dose were administered to a 25 g mouse. A total of 36 mice were used for each compound tested. Blood was collected by cheek bleeding at selected times between 0-168 h post-injection. Liver, spleen, kidney, brain and bone marrow were harvested at terminal time points (2, 8, 24, 48, 72 h). Tissue samples were collected and processed for bioanalysis. The drug concentration in blood and tissues was measured by LC-MS/MS using methods developed during preliminary studies and validated according to FDA guidance. Drug accumulation in tissue was assessed by calculating a tissue to plasma concentration ratio (Kp), an indicator of drug accumulation (FIGS. 6A, 6B, 7, and 38).

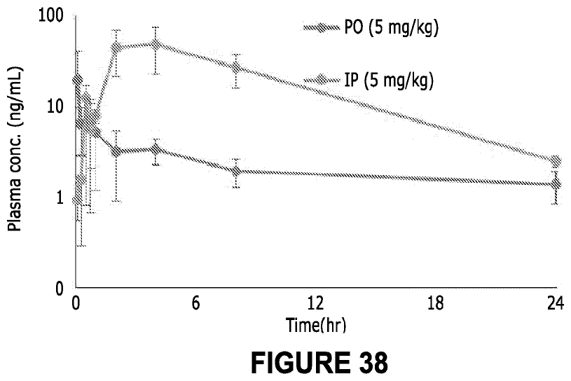

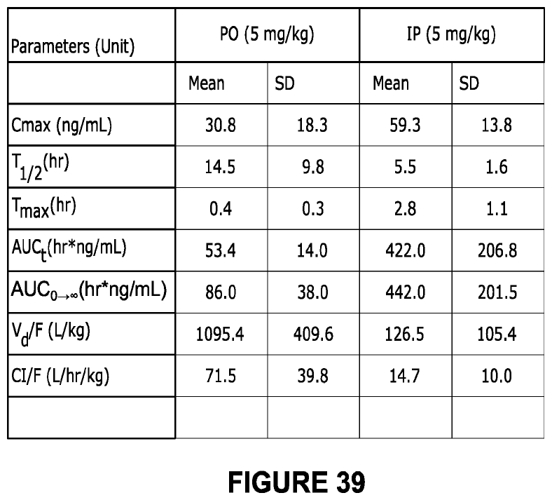

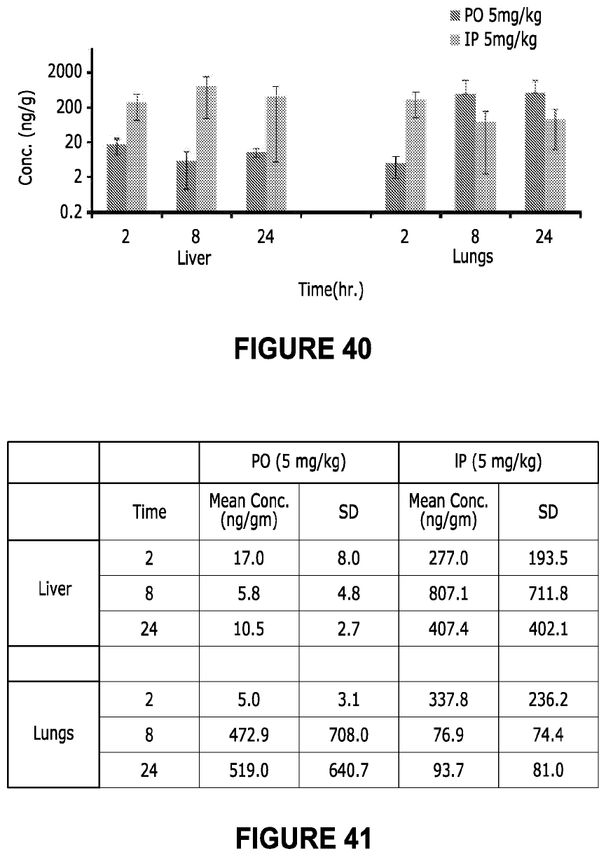

Pharmacokinetic parameter values were estimated from the plasma concentration-time data using standard noncompartmental methods and software (WinNonlin, PharSight Corp). The maximum plasma concentrations (Cmax), the time Cmax occurs (tmax), and the last plasma concentration measured, or the 24-hour concentration (C₂₄), were obtained directly from the individual plasma concentration-time profiles. Terminal elimination half-life (t1/2) was calculated by dividing 0.693 by terminal elimination rate constant Iz. Drug clearance (Cl) was calculated using standard equations. Area under the plasma concentration-time curve from 0 to 24 hours (AUCO-24) was calculated with the linear-log trapezoidal rule. The area under the plasma concentration-time curve from 0 to infinity (AUCO-infinity) was calculated as AUCO-24+C24/Iz. The absolute bioavailability (F) was calculated as the ratio between the AUCO-infinity from oral and intravenous routes using standard equations (FIG. 39).

Development of a physiological-based pharmacokinetic (PBPK) model. A global (whole-body) PBPK model is developed with data from plasma and tissue concentrations. The PBPK model fittings for drug plasma and tissue concentrations in mice are performed using ADAPT 5 (version 5, Biomedical Simulations Resource, Los Angeles, CA) as previously described (42). The PBPK model provides the framework for dosing strategies designed to achieve therapeutic drug concentrations deemed effective based on in vitro and pilot animal studies assessing efficacy.

### Determination of Binding Specificity and Characterization the Binding Epitope of PDS-0330 to Inhibit Colon Cancer Progression.

PDS-0330 is thought to have specific isoform binding to the C-terminus (CT) of human claudin-1. Without wishing to be bound by theory, this binding is believed to interfere with binding of Src to inhibit claudin-1 mediated cell functions.

This binding was examined in vitro using direct inhibitor binding assays against a panel of human claudins, then, using site-directed mutagenesis, probing the CT of claudin-1 to verify the PDS-0330 binding site and determine effects on Src, p-Akt and resistance to anoikis.

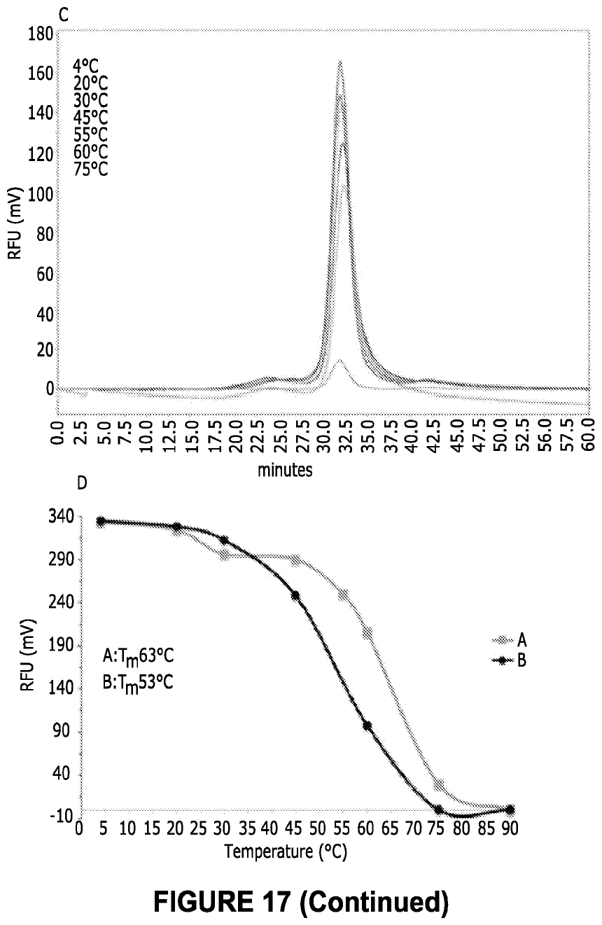

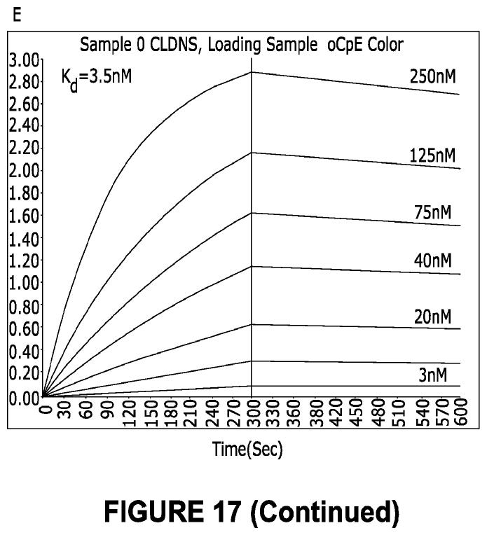

Data and Rationale: Data has been obtained illustrating: 1) expression and purification of human claudins, 2) monitoring of protein abundance, homogeneity, and stability during purification, and 3) assaying ligand and/or protein binding to human claudins via biophysical methods in vitro. Methods for expressing recombinant expression of numerous human claudins in HEK-293T or insect cells have been developed. Using eGFP-fusion or affinity-tagged constructs, the biochemical and biophysical behaviors of human claudins was monitored using SEC from impure (FIG. 17A) or pure samples (FIG. 17B). With fluorescence-detection size-exclusion chromatography (F-SEC), information about claudin abundance, oligomeric state, and homogeneity was obtained in a high-throughput manner (FIG. 17A) [Kewate and Gouaux, Structure, 2006 14(4)]. This data informs on claudin isoforms most amenable for in vitro study, and insect cell expression and subsequent affinity purification of these claudins shows that these molecules can be isolated with a high degree of purity and monodispersity (FIG. 17B). These two separate workflows allowed for a plethora of downstream biochemical or biophysical analyses; including denaturation temperature (Tm) determination (FIG. 17C&D), discovering small molecule ligands that bind and thermostabilized claudins (FIG. 17D), and/or protein interaction kinetic and affinity measurements (FIG. 17E).

PDS-0330 is thought to be selective to hCLDN-1 and its binding site resides on the C-terminal. Previously, using in silico methods, a model of the structure of hCLDN-1 was created then matched with 10 inhibitors (of which I-6 was initially identified) from a 100,000-compound library that bound to site 3, an intracellular cavity. Characterizing the molecular bases for PDS-0330 selective targeting of hCLDN-1 is required to better understand the mechanisms by which it inhibits colon cancer cell growth, and for rational drug design of new inhibitors with potentially greater selectivity and efficacy, based on a PDS-0330 scaffold. Combinatorial biochemical/biophysical techniques are useful to elucidate PDS-0330 binding epitope and its relation to isoform specificity.

### Determination of Binding Specificity of PDS-0330.

(i) hCLDN-1 and a panel of other human claudins are recombinantly expressed, including: hCLDN-2, −3, −4, −5, −6, −9, −11, −15, −17, −19 and −23. PDS-0330 binding directly to claudins is tested using F-SEC, thermal shift assays, microscale thermophoresis (MST), and bio-layer interferometry (BLI) as described herein. Briefly, claudins were expressed in HEK-293T and/or insect cells (Sf9, Tn5), solubilized out of membranes with detergents, and ultracentrifuged to remove insoluble debris. At this point, hCLDN-1 and other claudins can be further purified via affinity chromatography or used impure for direct binding analyses. These methodologies vary in precision. For less precise but direct binding characterization F-SEC-coupled thermal shift assays (FSEC-TS) can be used (45). Here, claudins with a C-terminal eGFP fusion were expressed, solubilized in detergent, and PDS-0330 added in vitro. The Tm without inhibitor was used as reference to test the degree of protein thermostabilization by the addition of PDS-0330. Direct binding of a molecule to the eGFP-tagged claudin resulted in stabilization and protection from thermal denaturation and increases in the Tm (FIG. 16D) (46). Fluorescence intensity plotted against temperature was used to generate Tm curves.

(ii) Direct binding measurements with higher precision are obtained with MST and BLI. The concept of MST is similar to FSEC-TS. Here, changes in claudin-eGFP fluorescence as a function of the concentration of non-fluorescent inhibitor-6 are monitored in solution during application of a temperature gradient (47). MST is used to measure binding affinities between claudins and PDS-0330, and for identifying different binding mechanisms based on analysis of unique thermophoresis signals. In addition, BLI is used to verify direct binding and measure the affinities and binding kinetics between claudins and inhibitor-6 (48). For this, PDS-0330 is biotinylated and immobilized on streptavidin biosensors then incubated with claudins at various concentrations (association) then buffer alone (dissociation). An inverse experiment is also run where histidine-tagged claudins are immobilized on nickel-NTA biosensors and PDS-0330 binding is assayed. Due to the complimentary yet non-overlapping data generated from the above methodologies direct binding to PDS-0330 to various claudins can be validated, verifying hCLDN-1 selectivity while obtaining information on binding constants, kinetics, and mechanisms. The ability to use impure preparations of proteins is a strength of this process, as it increases the throughput of binding characterization while maintaining experimental robustness due to the specificity unique to the probed fluorophore or chemically tagged molecule.

### Characterization of Binding Epitope of PDS-0330 Using Site Directed Mutagenesis.

(i) Guided by in silico docking experiments with PDS-0330 targeted to an intracellular site near the hCLDN-1 C-terminal (CT), site-directed mutagenesis is used to pinpoint the PDS-0330 binding epitope. CT mutants of hCLDN-1 were tested for their ability to maintain binding to PDS-0330 while characterizing affinity and kinetic changes using FSEC-TS, MST, and BLI. The CT of hCLDN-1 begins at the end of transmembrane helix (TM) 4, with a known palmitoylation site (49). Twenty-nine amino acids constitute the CT. The sequence is dominated by five basic residues, making the CT strongly positively charged. There is also a high potential for phosphorylation, as 10 of 29 residues are Thr, Ser, or Tyr. Without wishing to be bound by theory, it is thought that PDS-0330 binds CT through basic and polar interactions by recognition of a linear amino acid sequence and not via a binding pocket or cavity. Considering previous experiments that show simultaneous binding of ZO-1 and Src (Proto-oncogene tyrosine-protein kinase) to hCLDN-1 CT (9), it is thought that PDS-0330 binds the CT sequence RKTTSY or RPYPK. Both sites are upstream of the ZO-1 binding site and potential Src binding sites due to the presence of Tyr. Data shows that PDS-0330 inhibits Src association, which helps identify the binding site. By site-directed mutagenesis, the tyr residue is modified, the basic/polar side chains of these two sites to unequivocally identify the PDS-0330 binding epitope.

Determination if PDS-0330 Binds and Induce a Conformational Change to Regulate the Binding of Claudin-1 with Src.

PDS-0330 binding with claudin-1 is examined by analyzing proteolytic profiles of the protein in the presence of PDS-0330, a widely used method to determine binding of a small molecule to its target protein and to probe conformational changes of proteins (80). Direct binding of a small molecule to a protein can cause conformational changes that are accompanied by alteration in proteolysis patterns of the protein by proteases, such as trypsin. Cleavage conditions for claudin-1 protein are optimized, then a dose-dependent acceleration of trypsin cleavage of claudin-1 is performed with an EC50 (half maximal effective concentration) value. Positive results suggest that PDS-0330 binds to claudin-1 and enhances the accessibility of trypsin to claudin-1, presumably by changing claudin-1 conformation, thus abrogating its interaction with Src.

### Determination of the Efficacy of PDS-0330 in Inhibiting Claudin-1-Dependent Phenotypes in Model of Aggressive CRC.

Without wishing to be bound by theory, it is thought that PDS-0330 inhibits colon cancer progression and chemoresistance by inhibiting claudin-1 mediated cellular function. This can be studied using xenograft, orthotopic and GEM mouse models and PDOX to test the effect of PDS-0330 on tumor progression and metastasis.

Developing effective therapies against colon cancer metastasis is an urgent necessity as it can have significant bearing upon patients' survival. Data from numerous studies and current data strongly suggest that claudin-1 can be an excellent candidate for targeted therapy against CRC-metastasis. However, despite potential targetability, no therapeutic drug effective in inhibiting claudin-1 mediated CRC-malignancy is currently available or is in developmental stages. The present disclosure provides significant progress in this direction and has identified (and successfully tested) a second generation novel small molecule inhibitor, PDS-0330, effective against claudin-1 mediated fostering of “resistance to anoikis”, invasive mobility and chemoresistance in CRC cells and xenograft tumor growth in vivo. However, for future clinical application, extensive testing of this drug's efficacy and other analogs identified herein in models of CRC-progression and metastasis is required.

In vitro validation of the effects of PDS-0330 treatment. To validate that PDS-0330 effects are not cell-line specific as well as other promising analogs identified in aim 1, these studies are repeated using other claudin-1 expressing and non-expressing CRC cells. HT29, DLD-1, SW620 cells (claudin-1 expressing) and NCM460, IEC-6 (non-transformed cells) and SW620ShRNA cells (claudin-1 deficient) in resistance to anoikis and cell invasion. As control, CRC cells overexpressing claudin-2, also known to promote CRC (50) are used.

In vivo validation of the anti-tumorigenic and metastatic effects of 1-6 treatment: (i) Xenograft model. A decrease in xenograft tumor growth has been observed using SW80claudin-1 cells in mice subjected to PDS-0330 treatment (ip; 2.5, 5 mg/kg; Figure-19). These studies are elaborated using claudin-1 positive (HT29, DLD-1, SW620 cells) and negative (HCT116, SW620ShRNA) for similar xenograft tumor growth studies (15,50). Luciferin expressing cells (established in our lab) are used to help examining longitudinal growth of these tumors using live mouse imaging.

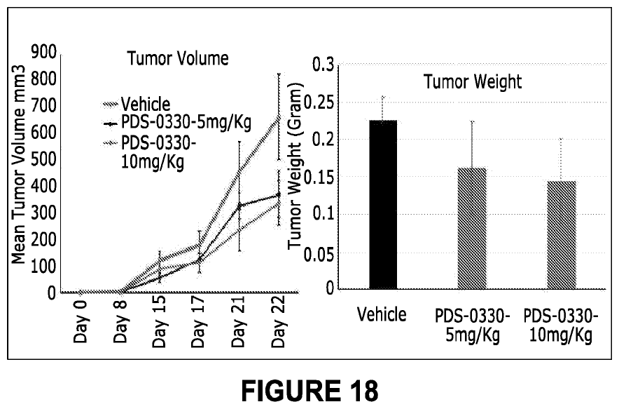

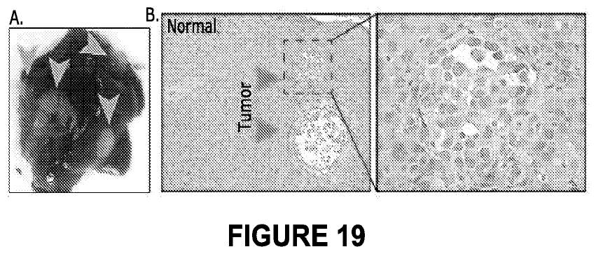

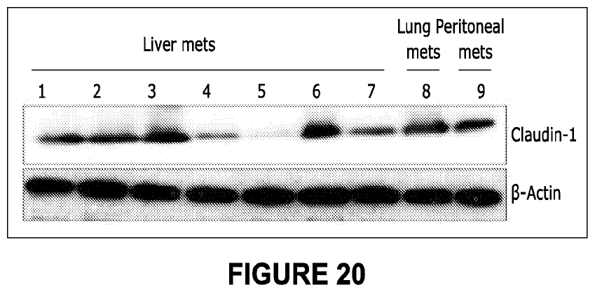

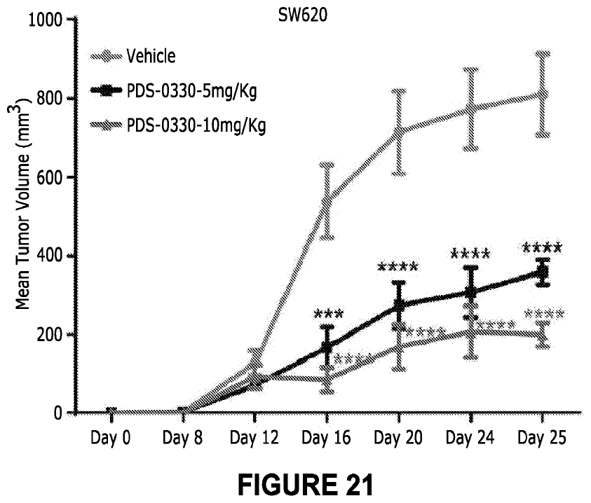

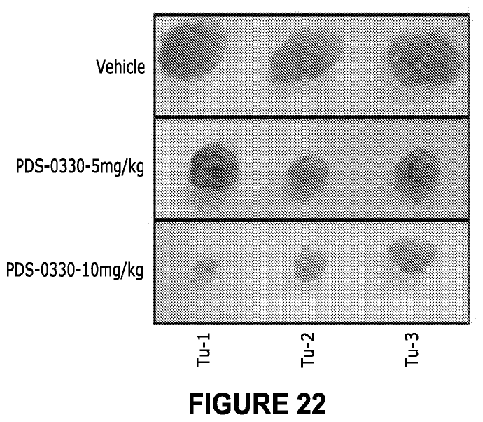

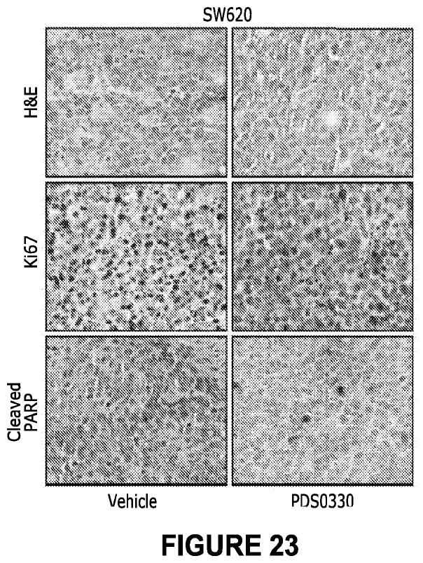

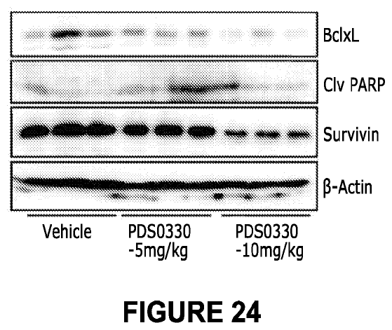

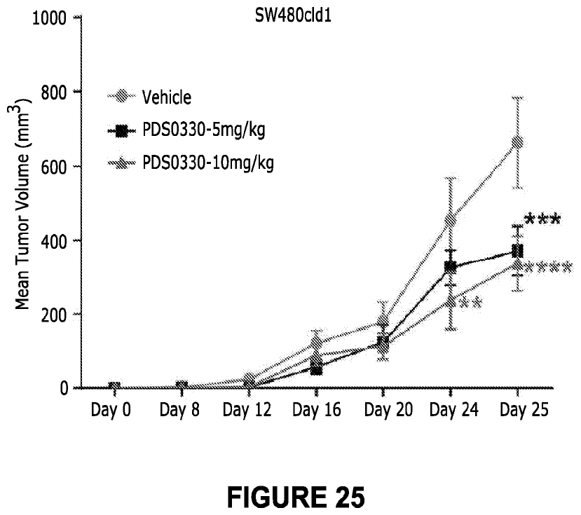

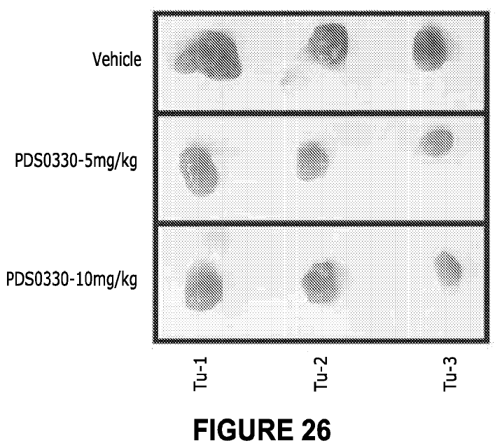

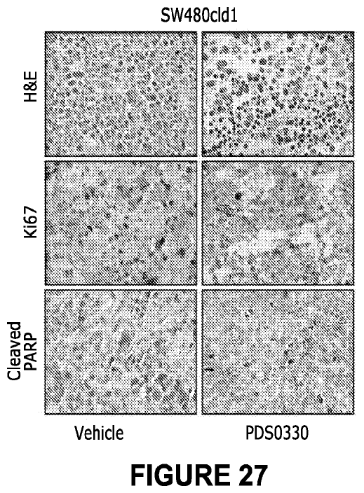

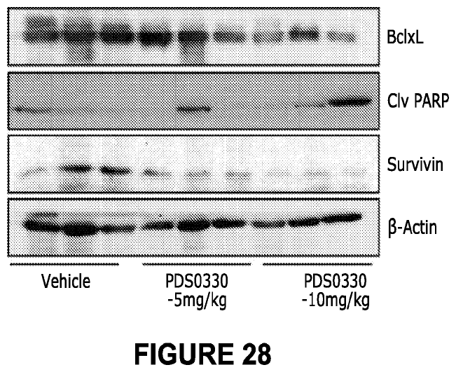

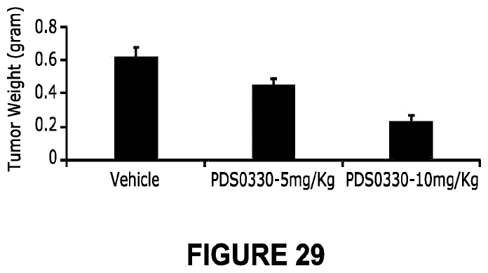

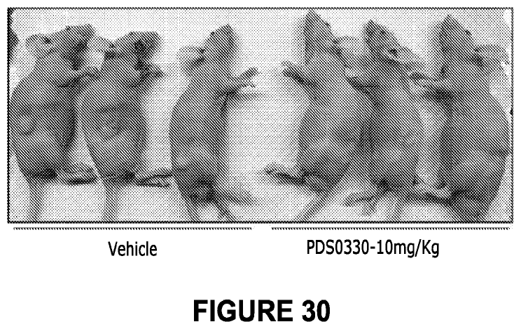

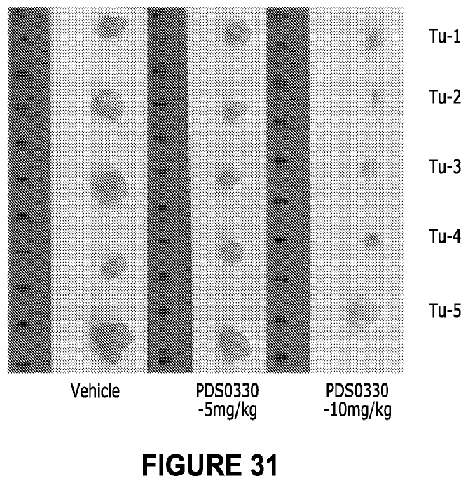

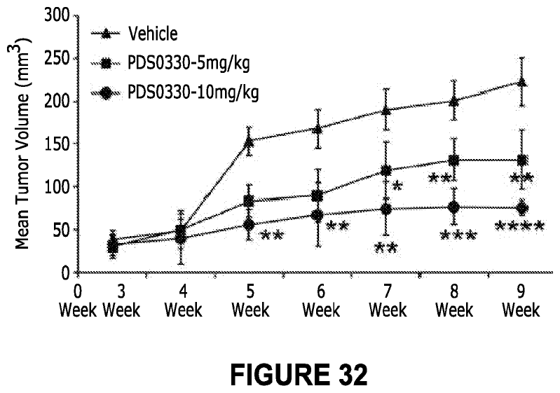

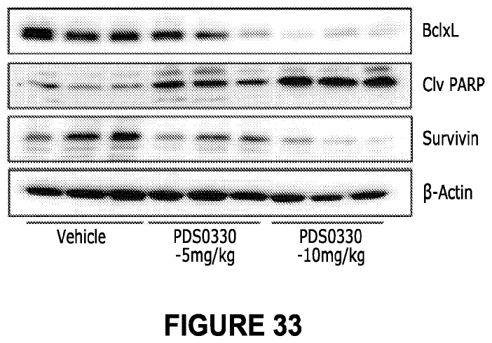

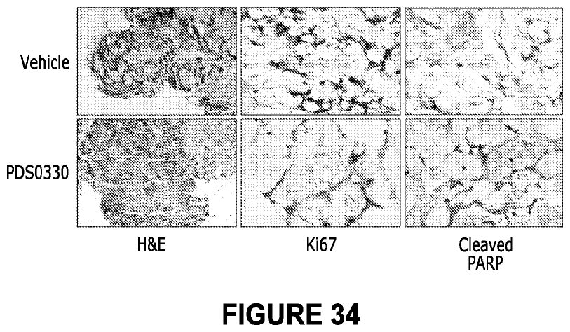

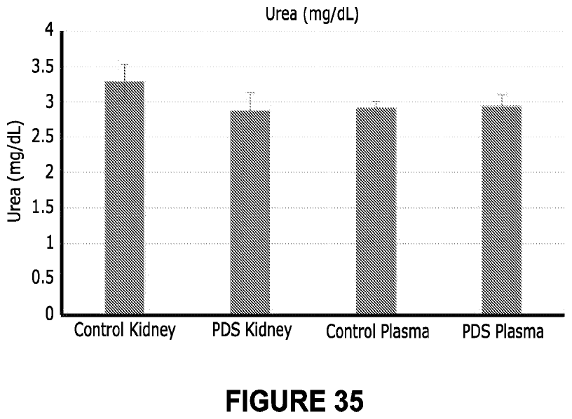

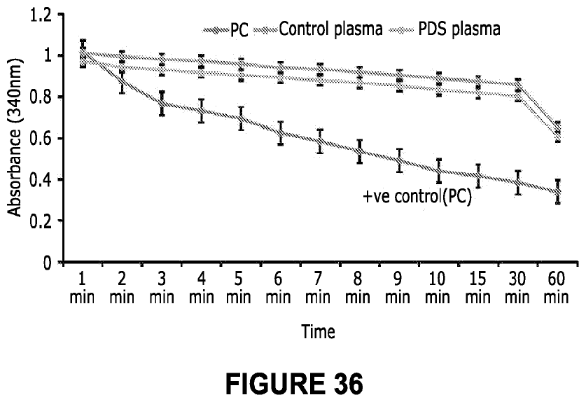

(ii) Orthotopic model. To evaluate the efficacy of PDS-0330 in inhibiting metastasis, a murine model of orthotopic cecal implantation of CRC metastasis was used, as it is the most relevant and unbiased animal model of CRC metastasis. These studies are routinely done in the lab (FIG. 20). SW480Claudin-1 (overexpressing claudin-1) and SW620 (high endogenous claudin-1) cells were used. To ensure efficient metastasis detection, these cells were engineered to express the firefly luciferase gene. Bioluminescence imaging (BLI) using the IVIS 200 imaging system was applied to monitor metastasis development. BLI is sensitive enough to detect microscopic metastases noninvasively. In brief, xenograft tumors (˜500 mm3) were excised, sectioned into 1 mm3 pieces and implanted into the subserosa of the cecum of athymic nude mice. It takes ˜5 weeks for CRC cells to start developing liver or lung metastasis, which was monitored by BLI. PDS-0330 was injected (i.p.; 2.5, 5 and 10 mg/kg based on preliminary data) at 2-3 weeks and effects were determined by bioluminescence imaging in the incidence and/or burdens of primary tumors and metastases between treated and untreated mice. Tissues from uninjected animals served as control for background fluorescence. Tumor Analysis: At the end of the experiments, primary tumors were excised, weighted and processed for the H&E, proliferative, apoptotic and stem cell marker analyses, expression of p-Src, Src, p-Akt, and association of claudin-1 and p-Src as described before. Liver and lungs were excised to validate metastasis using histological analyses. Pathological review is used to assess the degree of disease spread into the stroma. Tumors were classified as stage-I (localized growth), stage-II (local tumors+involvement of the parietal peritoneum, abdominal wall, diaphragm), stage-III (stage I, II+mesenteric lymph nodes) or stage-IV (stages I, II and/or III+liver and/or lung metastases) (FIG. 19).

(iii) Genetically engineered mice (GEM) and chemoresistant cells. Similar evaluations are performed using APCmin/cld-1 mice, a genetic model of claudin-1 mediated CRC progression (15). APCmin/cld-1 mice (7 weeks old; documented tumor incidence) are subjected to PDS-0330 treatment (5 or 10 mg/kg). Effects on tumor growth and invasion are determined. It is also determined if PDS-0330 treatment can overcome chemoresistance. For this purpose, chemoresistant CRC cells (DLD-1 and HT-29) established using sustained exposure to 5-FU treatment are used (Figure-16). Xenograft tumors are generated by implanting these cells in the dorsal flank of nude mice (n #10 mice/group). When tumors reach the size of ˜100 mm3, mice receive PDS-0330, 5—FU or PDS-0330+5-FU (at 5 mg/kg each drug; 4 times/week). At the end tumor analysis is done as in (ii).

### Determination of the Effect of PDS-0330 in Patient Derived Orthotopic Xenograft (PDOX) Model.

Male and female nu/nu mice aged 4-6 weeks undergo orthotopic implantation of human colon cancer tissue into the ascending colon or liver, respectively, using techniques known in the art (51,52). The tumors are allowed to grow for 2 weeks and then mice are given a single intraperitoneal dose (5 and 10 mg) of PDS-0330, with or without 5FU (10 mg). These studies use 5 CRC PDOX models and 5 CRC liver PDOX models. Data with these PDOX has demonstrated that most of them express high levels of claudin-1 (FIG. 20). Animals are sacrificed and tumor and metastatic tissues further analyzed as described above.

Establishment of fresh patient derived xenograft (PDX) with tumors from the operating room. Human patient colon cancers were excised under standard sterile conditions. Initially, tumor fragments were implanted subcutaneously within 30 min of patient surgery over the right and left upper and lower flanks in nude mice. Subcutaneous tumors were allowed to grow for 2-4 weeks until large enough to supply adequate tumor for orthotopic implantation for PDOX models.

Colon cancer PDOX models. Mice are anesthetized by intramuscular injection of 0.02 mL of the ketamine solution described above. The abdomen is prepped with betadine. An incision is then made vertically in the midline of the abdomen through the skin and peritoneum. The ascending colon is carefully exposed and two 1 mm3 PDX tumor piecesare attached onto the mesenteric border of the bowel wall using 8-0 surgical sutures. The bowel is then returned into the peritoneal cavity, and the abdominal wall and skin is closed with 6-0 absorbable surgical sutures.

Liver metastasis PDOX model. Colon-cancer liver metastases resected from patients are implanted in the liver of nude mice by cutting into 3 mm3 blocks. The left lobe of the liver of nude mice is exposed through a midline incision, and a single PDX metastasis fragment (3 mm3) is orthotopically implanted. The left lobe of the liver with the implanted metastasis is returned to the abdominal cavity, and the incision is closed in one layer using 6-0 nylon surgical sutures) (53-55). At autopsy of the PDOX models, all major organs are explored grossly for tumors or metastases. H&E staining and other sections are prepared for staining.

Statistics plan and power analysis for mouse numbers in each group. The above number of mice in each group is calculated using tumor data from previous studies and is based on the expectation of 96% power on a two-sample, two-sided t-test at the significance level of 5%. All western blot/cell culture experiments are repeated independently three times. A double blinded experimental design is used to avoid subject bias in mouse model studies. A two-sample t-test or the Wilcoxon rank sum test, is used to compare tumor size and/or metastasis distribution between groups. Longitudinal models, such as the linear mixed model, is employed to compare the trend of tumor growth over time. Kaplan-Meier analysis is used for survival analysis. In all analysis, a two-tailed P value of less than 0.05 is considered as statistically significant.

In view of the many possible embodiments to which the principles of the disclosure may be applied, it should be recognized that the illustrated embodiments are only examples and should not be taken as limiting the scope of the invention.

### REFERENCES

1. Adediran, S. A. et al. (2012) FEBS Lett. 586, 4186-4189
2. Longley, D. B. et al. (2005) The Journal of Pathology 205, 275-292
3. Zarour, L. R. et al. (2017) Cellular and molecular gastroenterology and hepatology 3, 163-173
4. Hammond, W. A. et al. (2016) Therapeutic advances in medical oncology 8, 57-84
5. Caruso, M. et al. (2014) Translational oncology 7, 456-463
6. Huo, Q. et al. (2009) Anticancer Research 29, 851-857
7. Dhawan, P. et al. (2005) The Journal of clinical investigation 115, 1765-1776
8. Kinugasa, T. et al. (2012) Anticancer Research 32, 2309-2314
9. Singh, A. B. et al. (2012) Carcinogenesis 33, 2538-2547
10. Paoli, P. et al. (2013) Biochimica et Biophysica Acta (BBA)-Molecular Cell Research 1833, 3481-3498
11. O'Brien, C. A. et al. (2006) Nature 445, 106
12. Boland, C. R. et al. (2016) The New England journal of medicine 374, 277-278
13. Assi, R. et al. (2016) Journal of gastrointestinal oncology 7, 284-297
14. Pope, J. L. et al. (2014) Gut 63, 622-634
15. Pope, J. L. et al. (2014) Molecular Cancer 13, 167
16. Croker, A. K. et al. (2008) Journal of cellular and molecular medicine 12, 374-390
17. Velasco-Velázquez, M. A. et al. (2011) The American journal of pathology 179, 2-11
18. Cho, R. W. et al. (2008) Current Opinion in Genetics & Development 18, 48-53
19. Zhou, Y. et al. (2017) Oncotarget 9, 33403-33415
20. Li, F. et al. (2006) Cell Research 17, 3
21. Skoda, J. et al. (2016) PloS one 11, e0159255-e0159255
22. Ennis, E. A. et al. (2015). ACS Chem. Neurosci. 6, 417-427
23. Witt, J. O. et al. (2016) Bioorg. Med. Chem. Lett. 26, 2481-2488
24. Jeffries, D. E. et al. (2016) Bioorg. Med. Chem. Lett. 26, 5757-5764
25. Wieting, J. M. et al. (2017) ACS Chem. Neurosci. 8, 1873-1879
26. Kharade, S. V. et al. (2018) Mol. Pharmacol. 94, 926-937
27. Rödl, C. B. et al. (2014) Eur. J. Med. Chem. 84, 302-311
28. Kodomari, M. et al. (2005) Tetrahedron Lett. 46, 5841-5843
29. Chekler, E. L. P. et al. (2008) Tetrahedron Lett. 49, 6709-6711
30. Clarysse, S. et al. (2011) Eur. J. Pharm. Sci. 43, 260
31. Chhonker, Y. S. et al. (2018) Journal of chromatography. B, Analytical technologies in the biomedical and life sciences 1072, 320-327
32. Chhonker, Y. S. et al. (2018) Journal of pharmaceutical and biomedical analysis 151, 84-90
33. Samad, M. B. et al. (2017) Macromolecular Bioscience 17, 1700096
34. Ebeid, K. et al. (2018) Nature nanotechnology 13, 72-81
35. Krishnaiah, M. et al. (2018) European Journal of Medicinal Chemistry 143, 936-947
36. Chhonker, Y. S. et al. (2018) Journal of Pharmaceutical and Biomedical Analysis 160, 202-211
37. Jang, G. R. et al. (2001) Medicinal Research Reviews 21, 382-396
38. Matsushima, H. et al. (1998) Drug Metabolism and Disposition 26, 240-245
39. Wan, H. et al. (2007) Journal of Liquid Chromatography & Related Technologies 30, 681-700
40. Huang, R. et al. (2006) Biochemical Pharmacology 71, 1695-1704
41. Huang, R. S. et al. (2008) Biomedicine & Pharmacotherapy 62, 59-64
42. Bi, Y. et al. (2015) The AAPS journal 18, 228-238
43. Miller, A. A. et al. (2009) Journal of clinical oncology: official journal of the American Society of Clinical Oncology 27, 1800-1805
44. Wang, X. et al. (2014) British journal of clinical pharmacology 78, 1005-1013
45. Hattori, M. et al. (2012) Structure (London, England: 1993) 20, 1293-1299
46. Rees, D. C. et al. (2001)Protein science: a publication of the Protein Society 10, 1187-1194
47. Jerabek-Willemsen, M. et al. (2014) Journal of Molecular Structure 1077, 101-113
48. Joy, C. et al. (2009) Combinatorial Chemistry & High Throughput Screening 12, 791-800
49. Van Itallie, C. M. et al. (2005) 118, Journal of Cell Science 1427-1436
50. Dhawan, P. et al. (2011) Oncogene 30, 3234-3247
51. Murakami, T. et al. (2015) PloS one 10, e0138752-e0138752
52. Murakami, T. et al. (2015) Journal of Surgical Oncology 112, 119-124
53. Murakami, T. et al. (2015) Journal of surgical oncology
54. Murakami, T. et al. (2015) PLOS One 10, e0138752
55. Kuo, T. H. et al. (1995) Proc Natl Acad Sci USA 92, 12085-12089

What is claimed:

1. A compound, or pharmaceutically acceptable salt thereof, having the structure of Formula I: 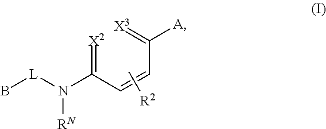
wherein
B is C₆-C₁₀ aryl or 5-10 membered heteroaryl comprising 1-3 ring heteroatoms independently selected from O, S, and N, wherein the aryl or heteroaryl is substituted with one R¹;
X¹ is CH or N;
X² is CH or N;
X³ is CH or N;
L is 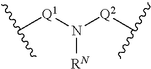
or 5-membered heteroaryl comprising 1 to 3 heteroatoms independently selected from O, S, and N;
Q¹ is a bond, C═O, or C═S;
Q² is C═O, C═S, or SO₂;
A is C₃-C₁₀ cycloalkyl, 3-10 membered heterocycloalkyl comprising 1-3 ring heteroatoms independently selected from O, S, and N, C₆-C₁₀ aryl, or 5-10 membered heteroaryl comprising 1-3 ring heteroatoms independently selected from O, S, and N, wherein the cycloalkyl, heterocycloalkyl, aryl, and heteroaryl are optionally substituted with 1-3 R³;
R¹, R², and R³ are each independently H, halo, OH, CN, C₁₋₆ alkyl, C₁₋₆ haloalkyl, C₁₋₆ hydroxyalkyl, C₁₋₆ alkoxy, or C₁₋₆ haloalkoxy; and
each R^{N} is independently H or C₁₋₆ alkyl,
or two R^{N} together with the atoms to which they are attached form a 5-6 membered heterocycloalkyl ring further comprising 0 or 1 additional ring heteroatoms selected from O, S, and N.

2. The compound or salt of claim 1, wherein B is

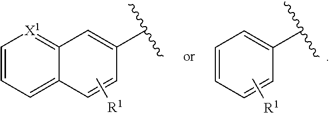

3. The compound or salt of claim 1 or 2, having a structure of Formula Ia:

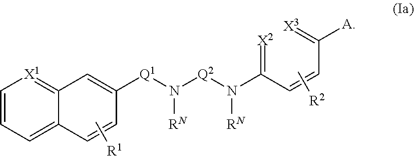

4. The compound or salt of any one of claims 1 to 3, wherein X¹ is CH.

5. The compound or salt of claim 1 or 2, having a structure of Formula Ib:

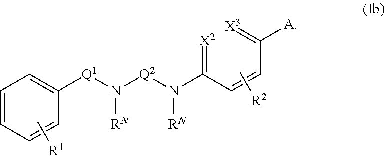

6. The compound or salt of any one of claims 1 to 5, wherein X² is CH.

7. The compound or salt of any one of claims 1 to 6, wherein X³ is CH.

8. The compound or salt of any one of claims 1 to 7, wherein Q¹ is a bond.

9. The compound or salt of any one of claims 1 to 7, wherein Q¹ is C═O.

10. The compound or salt of any one of claims 1 to 9, wherein Q² is C═O.

11. The compound or salt of any one of claims 1 to 9, wherein Q² is C═S.

12. The compound or salt of any one of claims 1 to 11, wherein A is 5-10 membered heteroaryl comprising 1-3 ring heteroatoms independently selected from O, S, and N optionally substituted with 1-3 R³.

13. The compound or salt of claim 12, wherein A is benzimidazole or benzoxazole.

14. The compound or salt of claim 12 or 13, wherein A is

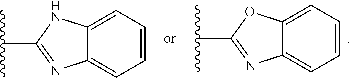 [SMILES: *c1nc2ccccc2[nH]1 ; *c1nc2ccccc2o1]

15. The compound or salt of any one of claims 12 to 14, wherein A is

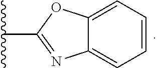 [SMILES: *c1nc2ccccc2o1]

16. The compound or salt of any one of claims 1 to 15, wherein R¹ is H or C₁₋₆ alkoxy.

17. The compound or salt of claim 16, wherein R¹ is H.

18. The compound or salt of claim 16, wherein R¹ is C₁₋₆ alkoxy.

19. The compound or salt of claim 18, wherein R¹ is methoxy.

20. The compound or salt of any one of claims 1 to 19, wherein at least one R^{N} is H.

21. The compound or salt of any one of claims 1 to 20, wherein each R^{N} is H.

22. A compound as recited in Table 1, or a pharmaceutically acceptable salt thereof.

23. A pharmaceutical composition comprising the compound or salt of any one of claims 1 to 22 and a pharmaceutically acceptable carrier or excipient.

24. A method of inhibiting claudin-1 comprising administering to a subject in need thereof a therapeutically effective amount of the compound or salt of any one of claims 1 to 22 or the pharmaceutical composition of claim 23.

25. A method of treating or preventing a disease or disorder capable of being modulated by claudin-1 inhibition, comprising administering to a subject in need thereof a therapeutically effective amount of the compound or salt of any one of claims 1 to 22 or the pharmaceutical composition of claim 23.

26. The method of claim 25, wherein the disease or disorder is hepatitis, diabetic nephropathy, ulcerative colitis, Crohn's disease, or cancer.

27. The method of claim 26, wherein the disease or disorder is cancer.

28. The method of claim 27, wherein the cancer is breast, colorectal, pancreatic, liver, nasopharyngeal, ovarian, uterine, lung, brain, skin, gastric, ovary, esophageal, prostate, colon, stomach, or oral squamous cell cancer.

29. The method of claim 28, wherein the cancer is colorectal cancer.

30. The compound or salt of any one of claims 1 to 22 or the pharmaceutical composition of claim 23 for use in inhibiting claudin-1.

31. The compound or salt of any one of claims 1 to 22 or the pharmaceutical composition of claim 23 for use in treating or preventing a disease or disorder capable of being modulated by claudin-1 inhibition.

32. The compound of claim 31, wherein the disease or disorder is hepatitis, diabetic nephropathy, ulcerative colitis, Crohn's disease, or cancer.

33. The compound of claim 32, wherein the disease or disorder is cancer.

34. The compound of claim 33, wherein the cancer is breast, colorectal, pancreatic, liver, nasopharyngeal, ovarian, uterine, lung, brain, skin, gastric, ovary, esophageal, prostate, colon, stomach, or oral squamous cell cancer.

35. The compound of claim 34, wherein the cancer is colorectal cancer.

36. Use of the compound or salt of any one of claims 1 to 22 or the pharmaceutical composition of claim 23 in the manufacture of a medicament for inhibiting claudin-1.

37. Use of the compound or salt of any one of claims 1 to 22 or the pharmaceutical composition of claim 23 in the manufacture of a medicament for treating or preventing a disease or disorder capable of being modulated by claudin-1 inhibition.

38. The use of claim 37, wherein the disease or disorder is hepatitis, diabetic nephropathy, ulcerative colitis, Crohn's disease, or cancer.

39. The use of claim 38, wherein the disease or disorder is cancer.

40. The use of claim 39, wherein the cancer is breast, colorectal, pancreatic, liver, nasopharyngeal, ovarian, uterine, lung, brain, skin, gastric, ovary, esophageal, prostate, colon, stomach, or oral squamous cell cancer.

41. The use of claim 40, wherein the cancer is colorectal cancer.
